(19) United States
(12) Patent Application Publication
(10) Pub. No.: US 2004/0258621 A1
(43) Pub. Date: Dec. 23, 2004

(54) Method of treating snoring and other obstructive breathing disorders

(76) Inventor: Warren Stern, Plymouth, MA (US)

Correspondence Address: ROPES & GRAY LLP, ONE INTERNATIONAL PLACE, BOSTON, MA, 02110-2624, US

(21) Appl. No.: 10/687,470

(22) Filed: Oct. 16, 2003

Related U.S. Application Data
(60) Provisional application No. 60/419,072, filed on Oct. 16, 2002.

(51) Int. Cl. A61L 9/04; A61K 38/54; A61K 31/557; A61K 31/366

(52) U.S. Cl. 424/45; 514/573; 514/460; 424/94.2

(57) ABSTRACT

A method of pharmaceutically managing snoring and impaired breathing is provided. This invention relates to treating snoring, sleep apnea, and other forms of sleep-disordered breathing in those with or without symptoms of or the diagnosis of gastro-intestinal reflux disease (GERD). It comprises administration of a therapeutically effective dose of Prevacid (Lansoprazole) or any other medication that can be used to treat symptoms of hyper-acidity or gastrointestinal reflux disease (GERD). The therapeutic medication may be used alone or in combination with other pharmacologic agents or mechanical modalities including but not limited to decongestants, antihistamines or mechanical nasal toilet. It also is of benefit in improving breathing disorders that are present while awake.

### REFERENCE TO RELATED APPLICATIONS

The present application claims priority to U.S. Provisional application 60/419,072, filed on Oct. 16, 2002, the entire content of which is incorporated herein by reference.

### FIELD OF THE INVENTION

The present invention relates to a method of treating snoring, sleep apnea, and other forms of sleep disordered breathing. It broadly concerns the management of snoring and sleep disordered breathing by reducing the incidence and/or magnitude thereof. More particularly, however, the present invention is directed to reducing and/or eliminating snoring and sleep disordered breathing by pharmacologic means, though it may be used as an adjunct to mechanical methods. Furthermore, it is of benefit while awake to improve compromised respiratory function

### BACKGROUND OF THE INVENTION

There is a significant incidence of snoring and obstructive breathing disorders in the general population. Snoring is a symptom of nocturnal upper airway obstruction. The sound is the repetitive resonance caused by soft tissue movement of the throat during sleep (uvula, soft palate, base of tongue, and throat walls). The major contributory cause of this noise is the oscillation of the thin edges of the soft palate (velum).

During inspiration a negative pressure is formed inside the chest cavity that sucks air into the lungs and simultaneously exerts pressure on the throat wall to collapse inward. This action is probably counterbalanced by intrinsic throat musculature that stabilizes and keeps this air passageway open.

A more pronounced collapse or obstruction can result in hypoxia, a condition in which airflow is reduced during inspiration with or without concomitant signs of hypoxemia. The condition of total functional collapse of the upper airway is referred to as obstructive sleep apnea (OSA). This condition is characterized by repeated episodes of an interrupted intake of air and arousal from sleep. Signs and symptoms

of this disorder are often characterized by excessive daytime drowsiness/sleepiness and cognitive disturbance with a significant potential for cardiovascular complications.

Some anatomical pharyngeal changes that have been observed in chronic snorers include: tongue enlargement, tonsillar and tonsillar pillar prominence, a drooping soft palate, and a narrowing of the back of the throat.

Exacerbating Factors

Positional—lying on ones back causes the tongue to fall backwards into the upper airway and narrows the passage

Age—as one ages, tissues tend to relax and fall back and obstruct the passage

Depth of sleep—Fatigue, drugs (tranquilizers, sleeping pills, and antihistamines), and alcohol a 11 act to relax the airway t issues and t end t o exacerbate airway collapse

Mouth breathing—path of higher resistance, putting collapsing pressure on the throat walls

Overweight

Late night eating

For many years, people have been seeking for the method for the treatment of snoring. Examples of general categories of devices which have been marketed to treat snoring include:

Keeping one off their backs

Keeping the tongue in place and the mouth shut

Extending the neck

Startling and waking the snorer

Examples of non-device treatments of snoring:

Sleep habit changes—cool and dark room—eliminating external noise—avoiding late night meals—avoiding stimulants—exercise—low pillow—elevating the head of the bed on 6″ blocks (also a treatment for esophageal reflux)

Alcohol use—avoid or diminish

Smoking—trying to eliminate the habit

Medicines—compound decongestants (avoid antihistamines)—saline and cortisone sprays—Acetazolamide (increases respiration by increasing blood acidity)—Theophylline (stimulates the respiratory center) Protriptyline—(inhibits REM sleep)

Weight loss

An external Nasal Dilator—keeps open the internal nasal valve

Mouth appliances

Continuous nasal positive airway pressure

Surgery—internal nasal, laser (to stiffen soft palate), and tonsillectomy

### OBJECT OF THE INVENTION

With the high incidence of snoring and obstructive sleep disorders in the general population and its associated functional problems, a pharmacologic ameliorating agent has distinct advantages over invasive or non-invasive methods presently used. An object of the present invention is to provide a method for the treatment of snoring, sleep apnea, and other forms of disordered breathing that reduces and/or eliminates some or all of the drawbacks of the methods known in the art.

### SUMMARY OF THE INVENTION

According to the present invention there is provided a method of treating snoring, sleep apnea and other forms of sleep disordered breathing by administering Prevacid (Lansoprazole) or any other medication that can be used to treat symptoms of hyper-acidity or gastro-intestinal reflux disease (GERD).

The invention also provides a method for treating respiratory impairment while awake, comprising the administration to a patient of a dose of Prevacid (Lansoprazole) or any other medication that can be used to treat symptoms of hyper-acidity or gastro-intestinal reflux disease (GERD).

The invention also provides a packaged pharmaceutical comprising: (i) a pharmaceutical preparation of an agent for treating symptoms of hyper-acidity or gastro-intestinal reflux disease and a pharmaceutically acceptable excipient, which preparation includes an amount of said agent(s) sufficient to reduce the symptoms or frequency of occurrence of sleep disordered breathing in a patient; and (ii) instructions for use of the preparation by a human patient for reducing the symptoms or frequency of occurrence of sleep disordered breathing.

The invention also provides the use of an agent for treating symptoms of hyper-acidity or gastro-intestinal reflux disease in the manufacture of a medicament for reducing the symptoms or frequency of occurrence of sleep disordered breathing in a human patient.

In one embodiment, the inhibitor is an H2 histamine receptor antagonists [e.g. TAGAMET™ (cimetidine), ZANTAC™ (ranitidine), PEPCID™ (famotidine), and AXID™ (nizatidine)]; an inhibitor of H⁺, K⁺ ATPase [e.g., PREVACID™ (lansoprazole), NEXIUM™ (esomeprazole magnesium), and PRILOSEC™ (omeprazole)]; Protonix (Wyeth), a Bismuth compound [e.g., PEPTO-BISMOL™ (bismuth subsalicylate) and DE-NOL™ (bismuth subcitrate)]; an antacid; a synthetic analog of somatostatin such as SANDOSTATIN™ (octreotide); an antiemetic agent (e.g., ZOFRAN™ (ondansetron), KYTRIL™ (granisetron hydrochloride); sucralfate; a prostaglandin analogs [e.g. CYTOTEC™ (misoprostol)]; a muscarinic cholinergic antagonist; a D2 antagonist (e.g., metoclopramide, trimethobenzamide and chlorpromazine); a chenodeoxycholic acid; an ursodeoxycholic acid; or a pancreatic enzyme preparations such as pancreatin and pancrelipase.

In one embodiment, the agent is an inhibitor of gastric secretion.

In one embodiment, the inhibitor is selected from the group consisting of H2 antagonists (e.g., Tagamet, Zantac, Pepcid, Axid) and proton pump inhibitors (such as Prilosec or Prevacid).

In one embodiment, the H2 histamine receptor antagonist selected from TAGAMET™ (cimetidine), ZANTAC™ (ranitidine), PEPCID™ (famotidine), or AXID™ (nizatidine).

In one embodiment, the H⁺, K⁺ ATPase selected from PREVACID™ (lansoprazole), NEXIUM™ (esomeprazole magnesium), or PRILOSEC™ (omeprazole). In a preferred embodiment, the inhibitor is PREVACID™ (lansoprazole).

In one embodiment, the inhibitor is PROTONIX® (pantoprazole sodium) or ACIPHEX® (rabeprazole sodium or pariprazole).

In one embodiment, the inhibitor is a compound or a pharmaceutical composition represented by any of formulas I-XVIII or salts thereof. These include all individual compounds or subclasses of compounds described under each and every formulas I-XVIII.

In one embodiment, the preparation further includes an anti-histamine.

In one embodiment, the preparation further includes a decongestant.

In one embodiment, the preparation further includes an anti-inflammatory agent.

In one embodiment, the invention is for reducing the occurrence or severity of snoring.

In one embodiment, the invention is for reducing the occurrence or severity of sleep apnea.

Another aspects of the invention provides a method for conducting a medical assistance reimbursement program, comprising: (i) providing a reimbursement program which permits, for prescription of an inhibitor of gastric secretion to reduce the symptoms or frequency of occurrence of sleep disordered breathing in a patient, at least partial reimbursement for said prescription to a healthcare provider or patient, or payment to a drug distributor for said prescription; (ii) processing one or more claims for prescription of said inhibtor for reducing the symptoms or frequency of occurrence of sleep disordered breathing; and (iii) reimbursing the healthcare provider or patient, or paying a drug distributor, at least a portion of the cost of said prescription.

It is contemplated that any of the above embodiments may be combined with other embodiments whenever appropriate.

### DETAILED DESRIPTION OF THE EXEMPLARY EMBODIMENTS

One aspect of the present invention provides a method for treating (eliminating, reducing the severity of, and/or reducing the frequency of occurrence of) sleep disordered breathing, by the administration to a patient of an agent that is otherwise used for treating symptoms of hyper-acidity or gastro-intestinal reflux disease, such as inhibitors of gastric secretion.

Another aspect of the invention provides a method for treating respiratory impairment in patients while they are awake, and includes the administration of an agent that is otherwise used for treating symptoms of hyper-acidity or gastro-intestinal reflux disease, such as inhibitors of gastric secretion.

Yet another aspect of the invention provides a packaged pharmaceutical that includes:

(i) a pharmaceutical preparation of an agent for treating symptoms of hyper-acidity or gastro-intestinal reflux disease and a pharmaceutically acceptable excipient, which preparation includes an amount of said agent(s) sufficient to reduce the symptoms or frequency of occurrence of sleep disordered breathing in a patient; and

(ii) instructions (written or pictorial) for use of the preparation by a human patient for reducing the symptoms or frequency of occurrence of sleep disordered breathing.

Still another aspect of the invention relates to the use of an agent for treating symptoms of hyper-acidity or gastro-intestinal reflux disease in the manufacture of a medicament for reducing the symptoms or frequency of occurrence of sleep disordered breathing in a human patient.

Another aspect of the invention relates to a method for conducting a medical assistance reimbursement program, comprising:

(i) providing a reimbursement program which permits, for prescription of an inhibitor of gastric secretion to reduce the symptoms or frequency of occurrence of sleep disordered breathing in a patient, at least partial reimbursement for said prescription to a healthcare provider or patient, or payment to a drug distributor for said prescription;

(ii) processing one or more claims for prescription of said inhibtor for reducing the symptoms or frequency of occurrence of sleep disordered breathing; and

(iii) reimbursing the healthcare provider or patient, or paying a drug distributor, at least a portion of the cost of said prescription.

While not meant to be limiting, it is noted that the subject methods and compositions can be used to reduce the occurrence or severity of snoring, as well as reduce the occurrence or severity of sleep apnea.

In certain preferred embodiments, the subject methods and pharmaceutical preparations use one or more agents that are inhibitors of gastric secretion. Such agents may include H2 antagonists and proton pump inhibitors. The agent may also be one which neutralizes gastric acid, or strengthens the gastroesophageal valve (such as metoclopramide (Reglan)).

Known pharmaceutical compositions which can be adapted for use in the subject methods and compositions include, but are not limited to, H2 histamine receptor antagonists [e.g. TAGAMET™ (cimetidine), ZANTAC™ (ranitidine), PEPCID™ (famotidine), and AXID™ (nizatidine)]; inhibitors of H⁺, K⁺ ATPase [e.g., PREVACID™ (lansoprazole), NEXIUM™ (esomeprazole magnesium), and PRILOSEC™ (omeprazole)]; Protonix (Wyeth), Bismuth compounds [e.g., PEPTO-BISMOL™ (bismuth subsalicylate) and DE-NOL™ (bismuth subcitrate)]; various antacids; synthetic analogs of somatostatin such as SANDOSTATIN™ (octreotide); antiemetic agents (e.g., ZOFRAN™ (ondansetron), KYTRIL™ (granisetron hydrochloride); sucralfate; prostaglandin analogs [e.g. CYTOTEC™ (misoprostol)]; muscarinic cholinergic antagonists; D2 antagonists (e.g., metoclopramide, trimethobenzamide and chlorpromazine); chenodeoxycholic acid; ursodeoxycholic acid; and pancreatic enzyme preparations such as pancreatin and pancrelipase.

The following section describes in detail about several compounds that may be adapted for use in the subject methods.

PRILOSEC™ (and its European equivalent LOSEC) (omeprazole): The active ingredient in Prilosec Delayed-Release Capsules is a substituted benzimidazole, 5-methoxy-2-[[(4-methoxy-3,5-dimethyl-2-pyridinyl)methyl]sulfinyl]-1H-benzimidazole, an inhibitor of gastric acid secretion. Its empirical formula is C₁₇H₁₉N₃O₃S, with a molecular weight of 345.42.

Prilosec is currently supplied on the market as delayed-release capsules for oral administration. According to the manufacturer, each delayed-release capsule contains either 10 mg or 20 mg of omeprazole in the form of enteric-coated granules with the following inactive ingredients: cellulose, disodium hydrogen phosphate, hydroxypropyl cellulose, hydroxypropyl methylcellulose, lactose, mannitol, sodium lauryl sulfate and other ingredients. The capsule shells have the following inactive ingredients: gelatin-NF, FD&C blue #1, FD&C red #40, D&C red #28, titanium dioxide, synthetic black iron oxide, isopropanol, butyl alcohol, FD&C blue #2, D&C red #7 calcium lake, and, in addition, the 10 mg capsule shell also contains D&C yellow #10.

The structure of PRILOSEC™ is listed below:

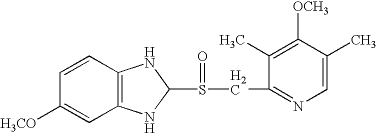

A genus of compounds encompassing the above described PRILOSEC™ composition, together with their various dosage forms and crystalline forms, may also be used, see U.S. Pat. Nos. 4,508,905, 4,786,505, 4,853,230, 6,147,103, 6,150,380, 6,166,213, and 6,191,148, European patent specification EP0005129B1, BE-898 880, and the patent applications EP-85850258,6, EP-A1-0 080 602, EP-0127 736, EP-0 134 400, EP-0 130 729, EP-0 150 586, DE-3415971, GB-2 082 580 and SE-A-8504048-3, all incorporated herein by reference. The genus of compounds that may be adapted for use in the instant invention are also described in more detail below in the NEXIUM™ section.

Briefly, the genus of compounds have the general structure represented by formula (V) (also see below)

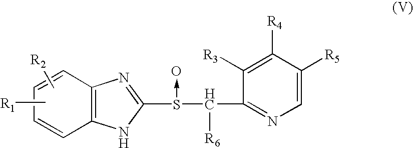

wherein R¹ and R² are the same or different and are each hydrogen, alkyl, halogen, carbomethoxy, carbethoxy, alkoxy, or alkanoyl, R⁶ is hydrogen, methyl or ethyl, R³, R⁴ and R⁵ are the same or different and are each hydrogen, methyl, methoxy, ethoxy, methoxyethoxy or ethoxyethoxy whereby R³, R⁴ and R⁵ are not all hydrogen, and whereby when two of R³, R⁴ and R⁵ are hydrogen the third of R³, R⁴ and R⁵ is not methyl. The compounds are potent gastric acid secretion inhibitors.

U.S. Pat. No. 6,150,380 describes a novel crystalline form of omeprazole (crystalline form A), which exhibits advantageous properties, such as being well-defined, being thermodynamically more stable and less hygroscopic than omeprazole form B, especially at room temperature. Omeprazole form A does also show a better chemical stability, such as thermo stability and light stability, than omeprazole form B. Since omeprazole form B can under certain conditions, completely or partly, be converted into omeprazole form A. Omeprazole form A is thereby characterized in being thermodynamically more stable than omeprazole form B. Omeprazole form A is further characterized as being essentially non-hygroscopic. The patent also provides a process for the preparation of omeprazole form A.

NEXIUM™ or ESOMEPRAZOLE MAGNESIUM: The active ingredient in NEXIUM™ (esomeprazole magnesium) Delayed-Release Capsules is bis(5-methoxy-2-[(S)-[(4-methoxy-3,5-dimethyl-2-pyridinyl)methyl]sulfinyl]-1H-benzimidazole-1-yl) magnesium trihydrate, a compound that inhibits gastric acid secretion. Esomeprazole is the S-isomer of omeprazole, which is a mixture of the S- and R-isomers. Its empirical formula is (C₁₇H₁₈N₃O₃S)₂Mg X 3 H₂O with molecular weight of 767.2 as a trihydrate and 713.1 on an anhydrous basis. The structural formula is:

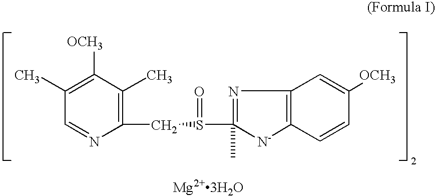

The magnesium salt is a white to slightly colored crystalline powder. It contains 3 moles of water of solvation and is slightly soluble in water. The stability of esomeprazole magnesium is a function of pH; it rapidly degrades in acidic media, but it has acceptable stability under alkaline conditions. At pH 6.8 (buffer), the half-life of the magnesium salt is about 19 hours at 25° C. and about 8 hours at 37° C. NEXIUM™ may be supplied as Delayed-Release Capsules for oral administration. Each delayed-release capsule may contain 20 mg or 40 mg of esomeprazole (present as 22.3 mg or 44.5 mg esomeprazole magnesium trihydrate) in the form of enteric-coated pellets with the following inactive ingredients: glyceryl monostearate 40-50, hydroxypropyl following inactive ingredients: glyceryl monostearate 40-50, hydroxypropyl acid copolymer type C, polysorbate 80, sugar spheres, talc, time and dose. The capsule shells may additionaly have the following inactive ingredients: gelatin, FD&C Blue #1, FD&C Red #40, D&C Red #28, titanium dioxide, shellac, ethyl alcohol, isopropyl alcohol, n-butyl alcohol, propylene glycol, sodium hydroxide, polyvinyl pyrrolidone, and D&C Yellow #10. For details, see product information sheet for NEXIUM™. According to the manufacturer AstraZeneca, NEXIUM™ is an improved version of PRILOSEC™ (and its European equivalent LOSEC) that reduces heartburn symptoms faster and has higher rates of healing than PRILOSEC™ for certain lesions caused by heartburn.

A genus of compounds encompassing the above described PRILOSEC™ and NEXIUM™ composition may also be used, see U.S. Pat. Nos. 4,045,563, and 4,738,974, and European patent specification EP0005129B1, all incorporated herein by reference.

The genus of compounds that may be adapted for use in the instant invention can be represented by formula II below:

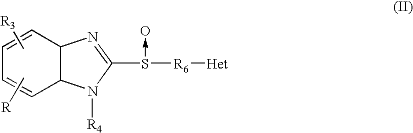

wherein R and R₃ are the same or different and are selected from the group consisting of hydrogen, alkyl, halogen, cyano, carboxy, carboxyalkyl, carboalkoxy, carboalkoxyalkyl, carbamoyl, carbamoyloxy, hydroxy, alkoxy, hydroxy alkyl, trifluoromethyl and acyl in any position, R₄ is selected from the group consisting of hydrogen, alkyl, acyl, carboalkoxy, carbamoyl, alkylcarbamoyl, dialkylcarbamoyl, alkylcarbonyl methyl, alkoxycarbonyl methyl and alkylsulphonyl, R₆ is selected from the group consisting of a straight or branched alkylchain having 1 to 4 carbon atoms, whereby only one methylene group is present between S and Het, and Het is selected from the group consisting of imidazolyl, imidazolinyl, benzimidazolyl, thiazolyl, thiazolinyl, quinolyl, piperidyl and pyridyl, which may be further substituted preferably in the 3 to 5 position with lower alkyl groups such as methyl, ethyl and propyl and/or with halo substituents such as chloro and bromo, or its therapeutically acceptable salts.

Alkyl R and R₃ of formula II are suitably alkyl having up to 7 carbon atoms, preferably up to 4 carbon atoms. Thus, alkyl R may be methyl, ethyl, n-propyl, isopropyl, n-butyl, isobutyl, or t-butyl.

Halogen R and R₃ are fluoro, iodo, bromo or chloro, preferably bromo or chloro.

Carboxy R and R₃ are the group HOOC—.

Carboalkyl R and R₃ are the groups HOOC-alkyl wherein the alkyl group has up to 4 carbon atoms, preferably up to 2 carbon atoms. Carboxyalkyl R and R3 are e.g. carboxymethyl, carboxyethyl.

Carboalkoxy R and R₃ are the groups alkyl-O—OC—, wherein the alkyl group has up to 4 carbon atoms, preferably up to 2 carbon atoms. Carboalkoxy R and R₃ are e.g. carbomethoxy (CH₃OOC—, carboethoxy (C₂H₅OOC—).

Carboalkoxy alkyl R and R₃ are the groups alkyl-OOC-alkyl, wherein the alkyl group has up to 4 carbon atoms, preferably up to 2 carbon atoms, and alkyl group has up to 4 carbon atoms, preferably up to 2 carbon atoms, such as carbomethoxymethyl (CH₃OOCCH₂), carbomethoxyethyl (CH₃OCC₂H₄—), carboethoxymethyl (C₂HOOCCH₂—) and carboethoxyethyl (C₂H₅OOCC₂H₄—).

Carbamoyl R and R₃ are the group H₂NCO—.

Carbamoylalkyl R and R₃ are the groups H₂NCO-alkyl, wherein the alkyl group has up to 4 carbon atoms preferably up to 2 carbon atoms, such as carbamoylmethyl (H₂NCOCH₂—), or carbamoylethyl (H₂NCOC₂H₄—).

Alkoxy R and R₃ are suitably alkoxy groups having up to 5 carbon atoms, preferably up to 3 carbon atoms, such as methoxy, ethoxy, n-propoxy, or isopropoxy.

Hydroxyalkyl R and R₃ have suitably up to 7 carbon atoms, preferably up to 4 carbon atoms and are straight or branched and are e.g. hydroxy methyl, 1-hydroxy-propyl-2,1-hydroxy-ethyl-2, or 1-hydroxy-2-methyl-propyl-2.

Acyl R and R₃ have preferably up to 4 c arbon atoms and are e.g. formyl, acetyl or propionyl.

Alkyl R₄ is a lower straight or branched alkyl group having up to 5 carbon atoms, preferably up to 3 carbon atoms, and is e.g. methyl, ethyl, or n-propyl.

Acyl R₄ has preferably up to 4 carbon atoms and is e.g. formyl, a cetyl or propionyl.

Carboalkoxy R₄ is the group alkyl-O—OC, wherein the alkyl group has up to 4 carbon atoms, preferably up to 2 carbon atoms, and is e.g. carbomethoxy (CH₃OOC—) or carboethoxy (C₂H₅OOC—).

Carbamoyl R₄ is the group H₂NCO—.

Alkylcarbamoyl R₄ is the group

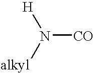

wherein the alkyl group may be straight or branched, has up to 4 carbon atoms, and is e.g. methylcarbamoyl, ethylcarbamoyl, or isopropylcarbamoyl.

Dialkylcarbamoyl R₄ is the group (alkyl)₂NCO wherein the alkyl groups each represent an alkyl group having up to 4 carbon atoms, and is e.g. dimethylcarbamoyl, diethylcarbarnoyl or N-methyl-N-ethylcarbanoyl.

Alkylcarbonylmethyl R₄ is the group alkyl-CO—CH2-, wherein the alkyl group has up to 4 carbon atoms, and is e.g. acetylmethyl or propionylmethyl.

Alkoxycarbonylmethyl R₄ is the group alkyl-O—CO—CH2-, wherein the alkyl group has up to 4 carbon atoms, and is e.g. methoxycarbonylmethyl, ethoxycarbonylmethyl or propoxycarbonylmethyl.

Alkylsulphonyl R₄ is the group alkyl-SO2- wherein the alkyl group has up to 4 carbon atoms, and is e.g. methylsulphonyl, ethylsulphonyl or isopropylsulphonyl.

Alkyl R₆ is a lower straight or branched alkyl having up to 4 carbon atoms and is e.g. methyl, (methyl)methyl or (ethyl)methyl, (isopropyl)methyl or (dimethyl)methyl.

The heterocyclic group Het, may be further substituted with alkyl or halogen preferably in the 3-5 position. Such alkyl groups are preferably lower alkyl groups such as methyl, ethyl or propyl. Such halogen substituents are preferably chloro or bromo.

The heterocyclic group Het is preferably bound in the 2-position, but may also be bound in the 4-position to the rest of the molecule.

Compounds of formula II above may be prepared according to the methods as described in U.S. Pat. No. 4,045,563, entire contents incorporated herein by reference.

Similar compounds are also described in BE-898 880, and patent applications EP-85850258,6, EP-A1-0 080 602, EP-0127 736, EP-0 134 400, EP-0 130 729, EP-0 150 586, DE-3415971 GB-2 082 580 and SE-A-8504048-3, all incorporated herein by reference. The last application describes 2-(2-disubstituted-aminobenzyl)sulfinyl benzimidazoles, e.g. 2-(2-dimethylaminobenzyl)sulfinyl benzimidazole, also called, NC-1300 and presented by Prof. S. Okabe at the Symposium on Drug Activity held on Oct. 17, 1985 in Nagoya, Japan, and which interacts with the H⁺ K⁺-ATPase after acid degradation within the parietal cells. (See for instance B. Wallmark, A. Brandstroom and H. Larson “Evidence for acid-induced transformation of omeprazole into an active inhibitor of H⁺ K⁺-ATPase within the partial cell”, Biochemica et Biophysica Acta 778, 549-558, 1984). Other compounds with similar properties are further mentioned in the patent U.S. Pat. No. 4,182,766 and the patent applications GB-2 141 429, EP-O 146 370 and GB-2 082 580 (incorporated herein by reference). A common feature of these compounds are that they are transformed into the biologically active compounds via rapid degradation/transformation in acid media.

Specifically, a compound or a therapeutically acceptable salt thereof is represented by formula II, in which R is selected from the group consisting of hydrogen, alkyl having up to four carbon atoms, halogen of the group consisting of fluoro, iodo, bromo and chloro, cyano, carboxy, carboxyalkyl in which the alkyl group has up to 4 carbon atoms, carboalkoxy in which the alkyl group has up to 4 carbon atoms, carboalkoxyalkyl wherein each of the alkyl groups has up to 4 carbon atoms, carbamoyl, carbamoylalkyl in which the alkyl group has up to 4 carbon atoms, hydroxy, alkoxy having up to 5 carbon atoms, hydroxyalkyl having up to 7 carbon atoms, trifluoromethyl and alkanoyl having up to 4 carbon atoms in any position; R3 is selected from the group consisting of hydrogen, alkyl having up to four carbon atoms, halogen of the group consisting of fluoro, iodo, bromo and chloro, carboxy, carboxyalkyl in which the alkyl group has up to 4 carbon atoms, carboalkoxy in which the alkyl group has up to 4 carbon atoms, carboalkoxyalkyl wherein each of the alkyl groups has up to 4 carbon atoms, carbamoyl, carbamoylalkyl in which the alkyl group has up to 4 carbon atoms, hydroxy, alkoxy having up to 5 carbon atoms, hydroxyalkyl having up to 7 carbon atoms, trifluoromethyl and alkanoyl having up to 4 carbon atoms in any position; R4 is selected from the group consisting of hydrogen, alkyl straight or branched chain having up to 5 carbon atoms, alkanoyl having up to 4 carbon atoms, carbamoyl, alkylcarbamoyl wherein the alkyl group may be straight or branched and has up to 4 carbon atoms, dialkylcarbamoyl wherein each alkyl group has up to 4 carbon atoms, alkylcarbonylmethyl wherein the alkyl group has up to 4 carbon atoms, alkoxycarbonylmethyl wherein the alkyl group has up to 4 carbon atoms, and alkylsulphonyl wherein the alkyl group has up to 4 carbon atoms; R6 is a straight or branched alkyl group having 1 to 4 carbon atoms, wherein at most one methylene group is present between S and Het, and Het is pyridyl, which may be further substituted preferably in the 3 to 5 position with lower alkyl groups such as methyl, ethyl or propyl or with halogen substituents such as chloro and bromo.

In one embodiment, R is hydrogen, hydroxy, cyano, methyl, ethyl, n-propyl, isopropyl, t-butyl, trifluoromethyl, methoxy, acetyl, carboxy, carbethoxy; R₃ is hydrogen, methyl or chloro; R₄ is hydrogen, methyl, carbamoyl, methylcarbamoyl, methylcarbonylmethyl, ethoxycarbonylmethyl or methylsulfonyl; R₆ is CH₂ and Het is 2-pyridyl, which may be further substituted with methyl, ethyl or chloro.

In one embodiment, R is hydrogen, methyl, ethyl trifluoromethyl, cyano or chloro; R₃ is hydrogen, methyl, ethyl, trifluoromethyl or chloro; R₄ is hydrogen; R₆ is CH(CH₃); and Het is 2-pyridyl, which may be further substituted with methyl, ethyl or chloro.

In one embodiment, R is hydrogen, methyl, ethyl, trifluoromethyl, cyano or chloro; R₃ is hydrogen, methyl, ethyl, trifluoromethyl or chloro; R₄ is hydrogen; R₆ is CH(C₂H₅); and Het is 2-pyridyl, which may be further substituted with methyl, ethyl or chloro.

In one embodiment, R is hydrogen, methyl, ethyl, trifluoromethyl, cyano or chloro; R₃ is hydrogen, methyl, ethyl, trifluoromethyl or chloro; R₄ is hydrogen; R₆ is CH; and Het is 2-pyridyl, which may be further substituted with methyl, ethyl or chloro.

More specifically, the following compounds or therapeutically acceptable salts thereof are within the scope of the invention:

2-[2-pyridylmethylsulfinyl]benzimidazole,

2-[2-pyridylmethylsulfinyl]-(4,6dimethyl)benzimidazole,

2-[2-pyridylmethylsulfinyl]-(5-ethyl)benzimidazole,

2-pyridylmethylsulfinyl]-(4-methyl, 6-chloro)benzimidazole,

2-[2-pyridylmethylsulfinyl]-(5-methoxy)benzimidazole,

2-[2-pyridylmethylsulfinyl]-(5-hydroxy)benzimidazole,

2-[2-pyridylmethylsulfinyl]-(5-acetyl)benzimidazole,

2-[2-pyridylmethylsulfinyl]-(5-carboxy)benzimidazole,

2-[2-pyridylmethylsulfinyl]-(5-carbethoxy)benzimidazole,

2-[2-(4-chloro)pyridyl-methylsulfinyl]benzimidazole),

2-[2-(5-methyl)pyridylmethylsulfinyl]benzimidazole,

2-[2-pyridylmethylsulfinyl]-N-methylbenzimidazole),

2-[2pyridyl-(methyl)methylsulfinyl]benzimidazole,

2-[2-pyridylmethylsulfinyl]-(4-methyl)benzimidazole,

2-[2-pyridylmethylsulfinyl]-(N-acetyl)benzimidazole,

2-[2-pyridylmethylsulfinyl]-(N-methoxycarbonyl)benzimidazole,

2-[2-pyridylmethylsulfinyl]-(5methyl)benzimidazole,

2-[2-pyridylmethylsulfinyl]-(5-chloro)benzimidazole,

2-[2-pyridylmethylsulfinyl]-(5-isopropyl)benzimidazole,

2-[2-pyridylmethylsulfinyl]-(5-t-butyl)benzimidazole,

2-[2-pyridylmethylsulfinyl]-(5-n-propyl)benzimidazole,

2-[2-pyridylmethylsulfinyl]-(N-carbamoyl)benzimidazole,

2-[2-pyridylmethylsulfinyl]-(N-methylcarbamoyl)benzimidazole,

2-[2-pyridylmethylsulfinyl]-(N-acetylmethyl)benzimidazole,

2-[2-pyridylmethylsulfinyl]-(N-ethoxycarbonylmethyl)benzimidazole,

2-[2-pyridylmethylsulfinyl]-(N-methylsulfonyl)benzimidazole,

2-[2(4-methyl)pyridylmethylsulfinyl]-(5-methyl)benzimidazole,

2-[2-(5-methyl)pyridylmethylsulfinyl]-(5-methyl)benzimidazole,

2-[2-pyridylmethylsulfinyl]-(6-chloro)benzimidazole,

2-[2-pyridyl-(ethyl)methylsulfinyl]benzimidazole,

2-[2-pyridyl-(ethyl)methylsulfinyl]-(5-chloro)benzimidazole,

2-[2-pyridyl-(methyl)methylsulfinyl]-5-ethyl)benzimidazole,

2-[2-(3-methyl)pyridylmethylsulfinyl]benzimidazole,

2-[2-(5-ethyl)pyridylmethylsulfinyl]-(5-methyl)benzimidazole,

2-[2-(5-ethyl)pyridylmethylsulfinyl]benzimidazole,

2-[2pyridyl-(ethyl)methylsulfinyl]-(5-ethyl)benzimidazole,

2-[2-pyridyl-(methyl)methylsulfinyl]-(5-methyl)benzimidazole,

2-[2-pyridyl-(methyl)methylsulfinyl]-(5-cyano)benzimidazole,

2[2pyridyl-(methyl)methylsulfinyl]-(5-trifluoro)benzimidazole,

2-[2-pyridyl-(ethyl)methylsulfinyl]-5-methyl)benzimidazole,

2-[2-pyridyl-(ethyl)methylsulfinyl]-(5-cyano)benzimidazole,

2-[2-pyridyl-(ethyl)methylsulfinyl]-(5-trifluoro)benzimidazole,

2-[2-pyridylmethylsulfinyl]-(4-chloro)benzimidazole,

2-[2-pyridyl-(isopropyl)methylsulfinyl]benzimidazole,

2-[2-pyridyl-(methyl)methylsulfinyl]-(5,6-dimethyl)benzimidazole, and

2-[2-pyridylmethylsulfinyl]-(5,6-dimethyl)benzimidazole.

It is also contemplated that pharmaceutical preparation of any of the above compounds, pharmaceutically acceptable non-toxic acid, neutral, or basic addition salt thereof in a therapeutically effective amount, or intermediates, are also within the scope of the invention.

More specifically, as described in U.S. Pat. Nos. 4,255,431, 4,337,257, and 4,508,905 (incorporated herein by reference), one class of the compounds that can be adapted for use in the instant invention is represented by formula III below:

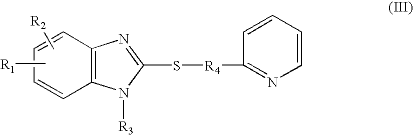

Another class of the compounds that can be adapted for use in the instant invention is represented by formula IV below:

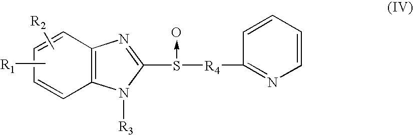

In each of these two formulas, R₁ and R₂ are each selected from the group consisting of hydrogen, alkyl, halogen, cyano, carboxy, carboxy-alkyl, carboalkoxy, carboalkoxyalkyl, carbamoyl, carbamoyl-oxy, hydroxy, alkoxy, hydroxyalkyl, trifluoromethyl and acyl in any position, R₃ is selected from the group consisting of hydrogen, alkyl, acyl, carboalkoxy, carbamoyl, alkylcarbamoyl, dialkylcarbamoyl, alkylcarbonylmethyl, alkoxycarbonylmethyl, and alkylsulphonyl, and R₄ is selected from the group consisting of straignt and branched alkylene groups having 1 to 4 carbon atoms, whereby at most one methylene group is present between S and the pyridyl group, and whereby the pyridyl group may be further substituted with alkyl or halogen.

A third, particularly preferred class of compounds are represented in formula V:

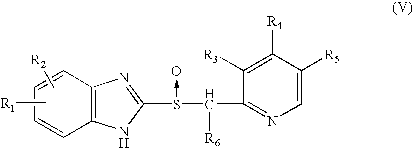

wherein R₁ and R₂ are the same or different, and are each selected from the group consisting of hydrogen, alkyl, halogen, carbomethoxy, carbethoxy, alkoxy, and alkanoyl, R₆ is selected from the group consisting of hydrogen, methyl, and ethyl, and R₃, R₄, and R₅ are same or different and are each selected from the group consisting of hydrogen, methyl, methoxy, ethoxy, methoxyethoxy and ethoxyethoxy, whereby R₃, R₄, and R₅ are not all hydrogen, and whereby when two of R₃, R₄, and R₅ are hydrogen, the third of R₃, R₄, and R₅ is not methyl.

Alkyl R₁ and R₂ of formula V are suitably alkyl having up to 7 carbon atoms, preferably up to 4 carbon atoms. Thus, alkyl R may be methyl, ethyl, n-propyl, isopropyl, n-butyl or isobutyl.

Halogen R₁ and R₂ is chloro, bromo, fluoro, or iodo.

Alkoxy R₁ and R₂ are suitably alkoxy groups having up to 5 carbon atoms, preferably up to 3 carbon atoms, as methoxy, ethoxy, n-propoxy, or isopropoxy.

Alkanoyl R₁ and R₂ have preferably up to 4 carbon atoms and are e.g., formyl, acetyl, or propionyl, preferably acetyl.

A preferred group of compounds of the general formula V are those wherein R₁ and R₂ are the same or different and are each selected from the group consisting of hydrogen, alkyl, carbomethoxy, alkoxy, and alkanoyl, whereby R₁ and R₂ are not both hydrogen, R₆ is hydrogen, and R₃, R₄, and R₅ are the same or different and are each selected from the group consisting of hydrogen, methyl, methoxy, and ethoxy, whereby R₃, R₄ and R₅ are not all hydrogen, and whereby when two or R₃, R₄, and R₅ are hydrogen the third of R₃, R₄, and R₅ is not methyl.

A second preferred group of compounds of the general formula V are those wherein R₁ and R₂ are the same or different and are each selected from the group consisting of hydrogen, alkyl, halogen, carbomethoxy, carbethoxy, alkoxy, and alkanoyl, R₆ is selected from the group consisting of hydrogen, methyl, and ethyl, R₃ is methyl, R₄ is methoxy, and R₅ is methyl.

A third preferred group of compounds of the general formula V are those wherein R₁ and R₂ are the same or different and are each selected from the group consisting of hydrogen, alkyl, halogen, carbomethoxy, carbethoxy, alkoxy and alkanoyl, R₆ is selected from the group consisting of hydrogen, methyl and ethyl, and R₃ is hydrogen, R₄ is methoxy and R₅ is methyl or R₃ is methyl, R₄ is methoxy and R₅ is hydrogen.

A fourth preferred group of compounds of the general formula V are those wherein R₁ and R₂ are the same or different and are each selected from the group consisting of hydrogen, alkyl, halogen, carbomethoxy, carbethoxy, alkoxy, and alkanoyl, R₆ is selected from the group consisting of hydrogen, methyl and ethyl, R₃ and R₅ are hydrogen and R₄ is methoxy.

A fifth preferred group of compounds of the general formula V are those wherein R₁ and R₂ are the same or different and are each selected from the group consisting of hydrogen, alkyl, halogen, carbomethoxy, carbethoxy, alkoxy, and alkanoyl, R₆ is selected from the group consisting of hydrogen, methyl and ethyl, and R₃ and R₅ are methyl and R₄ is hydrogen.

A sixth preferred group of compounds of the general formula V are those wherein R₁ and R₂ are the same or different and are each selected from the group consisting of hydrogen, alkyl, halogen, carbomethoxy, carbethoxy, alkoxy, and alkanoyl, R₆ is selected from the group consisting of hydrogen, methyl and ethyl, and R₃ and R₅ are hydrogen and R₄ is ethoxy, methoxyethoxy or ethoxyethoxy.

A seventh preferred group of compounds of the general formula V are those wherein R₁ and R₂ are the same or different and are each selected from the group consisting of hydrogen, alkyl, halogen, carbomethoxy, alkoxy, and alkanoyl, R₆ is selected from the group consisting of hydrogen, methyl, and ethyl, R₃, R₄, and R₅ are all methyl.

More specifically, in one embodiment, R₁, R₂, and R₆ have the meanings given in formula V (including the preferred group of compounds), R₃ is hydrogen, R₄ is methoxy, and R₅ is methyl; or R₅ is hydrogen, R₄ is methoxy, and R₃ is methyl.

In one embodiment, R₁, R₂, and R₆ have the meanings given in formula V (including the preferred group of compounds), R₃ and R₅ are methyl, and R₄ is methoxy.

In one embodiment, R₁, R₂, and R₆ have the meanings given in formula V (including the preferred group of compounds), R₃ and R₅ are hydrogen, and R₄ is methoxy, ethoxy, methoxyethoxy, or ethoxyethoxy.

In one embodiment, R₁, R₂, and R₆ have the meanings given in formula V (including the preferred group of compounds), R₃ and R₅ are methyl, and R₄ is hydrogen.

In one embodiment, the useful compounds or therapeutically acceptable salts thereof are selected from the group consisting of:

2-[2-(3,4-dimethyl)-pyridylmethylsulfinyl]-(5-acetyl-6-methyl)-benzimidazole;

2-[2-(3,5-dimethyl)-pyridylmethylsulfinyl]-(4,6-dimethyl)-benzimidazole;

2-[2-(4,5-dimethyl)-pyridylmethylsulfinyl]-(5-carbomethoxy)-benzimidazole;

2-[2-(4,5-dimethyl)-pyridylmethylsulfinyl]-(5-acetyl-6-methyl)-benzimidazole;

2-[2-(4,5-dimethyl)-pyridylmethylsulfinyl]-(5-carbomethoxy-6-methyl)-benzimidazole;

2-[2-(3,4-dimethyl)-pyridylmethylsulfinyl]-(5-carbomethoxy-6-methyl)-benzimidazole;

2-[2-(3,5-dimethyl)-pyridylmethylsulfinyl]-(5-acetyl-6-methyl)-benzimidazole;

2-[2-(3,4,5-trimethyl)-pyridylmethylsulfinyl]-(5-acetyl-6-methyl)-benzimidazole;

2-[2-(4-methoxy)-pyridylmethylsulfinyl]-(5-acetyl-6-methyl)-benzimidazole;

2-[2-(4-methoxy)-pyridylmethylsulfinyl]-(4,6-dimethyl)-benzimidazole;

2-[2-(3,5-dimethyl-4-methoxy)-pyridylmethylsulfinyl]-(5-acetyl-6-methyl)-benzimidazole;

2-[2-(3,5-dimethyl)-pyridylmethylsulfinyl]-(5-carbomethoxy-6-methyl)-benzimidazole;

2-[2-(3,4,5-trimethyl)-pyridylmethylsulfinyl]-(5-carbomethoxy-6-methyl)-benzimidazole;

2-[2-(4-methoxy)-pyridylmethylsulfinyl]-(5-carbomethoxy-6-methyl)-benzimidazole;

2-[2-(4-ethoxy)-pyridylmethylsulfinyl]-(5-carbomethoxy-6-methyl)-benzimidazole;

2-[2-(3-methyl-4-methoxy)-pyridylmethylsulfinyl]-(5-carbomethoxy-6-methyl)-benzimidazole;

2-[2-(3,5-dimethyl-4-methoxy)-pyridylmethylsulfinyl]-(5-carbomethoxy-6-methyl)-benzimidazole;

2-[2-(4-methoxy-5-methyl)-pyridylmethylsulfinyl]-(5-carbomethoxy-6-methyl)-benzimidazole;

2-[2-(3,5-dimethyl)-pyridylmethylsulfinyl]-(5-carbomethoxy)-benzimidazole;

2-[2-(3,5-dimethyl-4-methoxy)-pyridylmethylsulfinyl]-(5-carbomethoxy)-benzimidazole;

2-[2-(3,5-dimethyl-4-methoxy)-pyridylmethylsulfinyl]-(5-acetyl)-benzimidazole;

2-[2-(4-methoxy-5-methyl)-pyridylmethylsulfinyl]-(5-methoxy)-benzimidazole;

2-[2-(3,5-dimethyl-4-methoxy)-pyridylmethylsulfinyl]-(5-methoxy)-benzimidazole;

2-[2-(3,5-dimethyl-4-methoxy)-pyridylmethylsulfinyl]-(5-methyl)-benzimidazole;

2-[2-(3,5-dimethyl-4-methoxy)-pyridylmethylsulfinyl]-benzimidazole;

2-[2-(3,5-dimethyl-4-methoxy)-pyridylmethylsulfinyl]-(5-chloro)-benzimidazole;

It is also contemplated that pharmaceutical preparation of any of the above compounds, pharmaceutically acceptable non-toxic acid, neutral, or basic addition salt thereof in a therapeutically effective amount, or intermediates, are also within the scope of the invention.

Depending on the process conditions and the starting materials, the end product is obtained either as the free base or in the acid addition salt, both of which are included within the scope of the invention. That is, the therapeutically acceptable salts of any of the compounds described above are also included within the scope of the invention. Thus, basic, neutral or mixed salts may be obtained as well as hemi, mono, sesqui or polyhydrates. The acid addition salts of the new compounds may in a manner known per se be transformed into free base using basic agents such as alkali or by ion exchange. On the other hand, the free bases obtained may form salts with organic or inorganic acids. In the preparation of acid addition salts preferably such acids are used which form suitable therapeutically acceptable salts. Such acids include hydrohalogen acids, sulfonic, phosphoric, nitric, and perchloric acids; aliphatic, alicyclic, aromatic, heterocyclic carboxy or sulfonic acids, such as formic, acetic, propionic, succinic, glycolic, lactic, malic, tartaric, citric, ascorbic, maleic, hydroxymaleic, pyruvic, phenylacetic, benzoic, p-aminobenzoic, antranilic, p-hydroxybenzoic, salicylic or p-aminosalicylic acid, embonic, methanesulfonic, ethanesulfonic, hydroxyethane-sulfonic, ethylenesulfonic, halogenbenzenesulfonic, toluenesulfonic, naphtylsulfonic or sulfanilic acids; methionine, tryptophane, lysine or arginine.

These or other salts of the new compounds, as e.g. picrates; may serve as purifying agents of the free bases obtained. Salts of the bases may be formed, separated from solution, and then the free base can be recovered from a new salt solution in a purer state. Because of the relationship between the new compounds in free base form and their salts, it will be understood that the corresponding salts are included within the scope of the invention.

Some of the new compounds may, depending on the choice of starting materials and process, be present as optical isomers or racemate, or if they contain at least two asymmetric carbon atoms, be present as an isomer mixture (racemate mixture).

The isomer mixtures (racemate mixtures) obtained may be separated into two stereoisomeric (diastereomeric) pure racemates by means of chromatography or f ractional crystallization.

The racemates obtained can be separated according to known methods, e.g. recrystallization from an optically active solvent, use of microorganisms, reactions with optically active acids forming salts which can be separated, separation based on different solubilities of the diastereomers. Suitable optically active acids are the L- and D-forms of tartaric acid, di-o-tolyl-tartaric acid, malic acid, mandelic acid, camphorsulfonic acid or quinic acid, Preferably the more active part of the two antipodes is isolated.

The starting materials are known or may, if they should be new, be obtained according to processes known per se.

The synthesis of the compounds represented by formula V, various salts of the compounds, isomers, racemates, derived pharmaceutical compositions, suitable administration routes, dosage forms, and samples of comppound synthesis can all be found in EP 0005129B1, the entire content of which is incorporated herein by reference.

In one embodiment, the compound known under the generic name omeprazole, having the structural formula VI (described below, also described in European patent specification 0005129) may be adapted for use in the subject mathod.

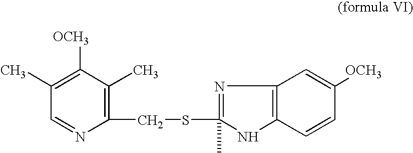

The term “omeprazole” as used in this specification designates the neutral form of the compound of the formula VI, that is the form as given in the formula VI without salt forming components present. A problem with omeprazole is its stability characteristics. Upon storage without any special precautions being taken, it is degraded at a rate which is higher than desired. A storage during accelerated conditions, that is at +37° C. and at a relative humidity of 80% for a period of 6 months, about 6% of the substance is converted to degradation products. While the rate of decomposition of omeprazole at normal storage conditions is lower, it is nevertheless desirable to obtain physical forms of omeprazole which exhibit improved stability.

New forms of omeprazole which exhibit improved storage stability is described below. It has been found that the novel alkaline salts of omeprazole with the structural formula VII below:

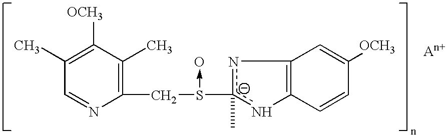

wherein n is 1, 2, or 4; Aⁿ⁺ is Li⁺, Na⁺, K⁺, Mg²⁺, Ca²⁺, Ti⁴⁺, N⁺(R₁)₄ or

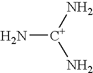

wherein R₁ is an alkyl group containing 1-4 carbon atoms are more stable during storage than the corresponding neutral form of omeprazole. The salts of the formula VII are also easier to handle than the neutral form in the manufacture of pharmaceutical dosage units.

A preferred group of omeprazole salts of the formula VII are those wherein Aⁿ⁺ is Na⁺, K⁺, Mg²⁺ and Ca²⁺.

Further preferred salts are those wherein Aⁿ⁺ is Na⁺, Mg²⁺ and Ca²⁺. The Na⁺-salt is especially preferred for the preparation of liquid pharmaceutical formulations, e.g. solutions for intravenous administration. The Mg²⁺ and Ca²⁺ salts are especially preferred for the preparation of tablets. The Mg²⁺ salt is particularly preferred.

Illustrative examples of the alkyl group R₁ are CH₃, C₂H₅, n-C₃H₇, and n-C₄H₉.

The novel salts I of the invention are prepared by reacting omeprazole of the formula:

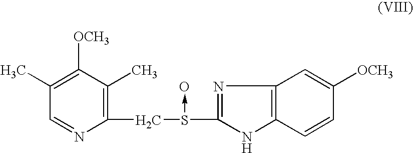

with a base capable of releasing the cation

Aⁿ⁺

wherein Aⁿ⁺ is as defined above, to give a salt of the formula

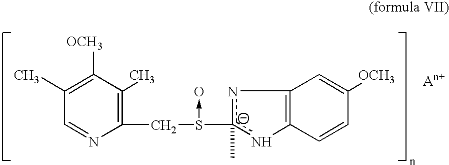

which salt is thereafter isolated.

Examples of bases capable of releasing the cation Aⁿ⁺, and examples of reaction conditions are given below.

(a) Salts of the formula a wherein A is Li, Na or K are prepared by treating omeprazole with LiOH, NaOH or KOH in an aqueous or nonaqueous medium or with LiOR, LiNH₂, LiNR₂, NaOR, NaNH₂, NaNR₂, KOR, KNH₂ or KNR₂, wherein R is an alkyl group containing 1-4 carbon atoms, in a nonaqueous medium.

(b) Salts of the formula VII wherein A is Mg, Ca, or Ti are prepared by treating omeprazole with Mg(OR)₂, Ca(OR)₂, CaH₂, Ti(OR)₄ or TiH₄, wherein R is an alkyl group containing 1-4 carbon atoms, in a nonaqueous solvent such as an alcohol (only for the alcoholates), e.g. ROH, or in an ether such as tetrahydrofuran.

(c) Salts of the formula VII wherein A is

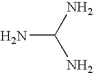

are prepared by treating omeprazole with the strong base

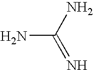

dissolved in a solvent, for example, an alcohol.

(d) A salt of formula VII may be converted to another salt of the same formula by exchanging the cation. When both the starting material and the salt obtained as final product are sufficiently soluble, such an exchange may be performed by using a cation-exchange resin saturated with the cation desired in the product. The exchange may also be performed by utilizing the low solubility of a desired salt. By this principle, for example, Na⁺ as a counter ion may be exchanged for Ca²⁺ or Mg²⁺.

(e) The reaction between the compounds (i) and (ii) may also be carried out by ion-pair extraction. For example, tetrabutylammonium salts of the invention may be prepared by dissolving the Na⁺-salt in water containing tetrabutylammonium sulfate followed by extraction of the tetrabutylammonium salt I into a methylene chloride phase, and subsequent isolation of the tetrabutylammonium salt I. In this manner also other tetraalkylammonium salts I may be prepared.

Illustrative examples of the radical R are CH₃, C₂H₅, n-C₃H₇, n-C₄H₉, i-C₄H₉, sec.-C₄H₉ and tert.-C₄H₉.

The preparation of various omeprazole salts, such as sodium, potassium, calcium, magnesium salts, can be found in U.S. Pat. No. 4,738,974, the entire content incorporated herein by reference.

U.S. Pat. No. 4,786,505 (incorporated herein by reference) further describes pharmaceutical preparation containing omeprazole together with an alkaline reacting compound or an alkaline salt of omeprazole, optionally together with an alkaline compound as the core material, one or more subcoating layers comprising inert reacting compounds which are soluble or rapidly disintegrating in water, or polymeric, water soluble film forming compounds, optionally containing pH-buffering alkaline compounds and an enteric coating as well as a process for the preparation thereof and the use in the treatment of gastrointestinal diseases. All these compositions can be adapted for use in the instant invention.

Specifically, the omeprazole core is mixed with inert, preferably water soluble, conventional pharmaceutical constituents to obtain the preferred concentration of omeprazole in the final mixture and with an alkaline reacting, otherwise inert, pharmaceutically acceptable substance (or substances), which creates a “micro-pH” around each omeprazole particle of not less than pH=7, preferably not less than pH=8, when water is adsorbed to the particles of the mixture or when water is added i n small amounts to the mixture. Such substances can be chosen among, but are not restricted to substances such as the sodium, potassium, calcium, magnesium and aluminium salts of phosphoric acid, carbonic acid, citric acid or other suitable weak inorganic or organic acids; substances normally used in antacid preparations such as aluminium, calcium and magnesium hydroxides; magnesium oxide or composite substances, such as Al₂O₃.6MgO.CO₂.12H₂O, (Mg₆Al₂(OH)₁₆CO₃.4H₂O), MgO.Al₂O₃.2SiO₂.nH₂O (n is an integer no less than2) or similar compounds; organic pH-buffering substances such as trihydroxymethylaminomethane or other similar, pharmaceutically acceptable pH-buffering substances. The stabilizing, high pH-value in the powder mixture can also be achieved by using an alkaline reacting salt of omeprazole such as the sodium, potassium, magnesium, calcium etc. salts of omeprazole, which are described in e.g. EP-A2-No. 124,495, either alone or in combination with a conventional buffering substance as previously described.

The powder mixture is then formulated into small beads i.e. pellets, tablets, hard gelatine or soft gelatine capsules by conventional pharmaceutical procedures. The pellets, tablets or gelatin capsules are used as cores for further processing.

The omeprazole containing alkaline reacting cores must be separated from the enteric coating polymer(s) containing free carboxyl groups, which otherwise causes degradation/discolouration of omeprazole during the coating process or during storage. The subcoating layer, in the following defined as the separating layer, also serves as a pH-buffering zone in which hydrogen ions diffusing from the outside in towards the alkaline core can react with hydroxyl ions diffusing from the alkaline core towards the surface of the coated articles. The pH-buffering properties of the separating layer can be further strengthened by introducing in the layer substances chosen from a group of compounds usually used in antacid formulations such as, for instance, magnesium oxide, hydroxide or carbonate, aluminium or calcium hydroxide, carbonate or silicate; composite aluminium/magnesium compounds such as, for instance Al₂O₃.6MgO.CO₂.12H₂O, (Mg₆Al₂(OH)₁₆CO₃.4H₂O), MgO.Al₂O₃.2SiO₂.nH₂O (n is an integer no less than 2), or similar compounds; or other pharmaceutically acceptable pH-buffering compounds such as, for instance the sodium, potassium, calcium, magnesium and aluminium salts of phosphoric, citric or other suitable, weak, inorganic or organic acids.

The separating layer consists of one or more water soluble inert layer, optionally containing pH-buffering compounds. The separating layer(s) can be applied to the cores—pellets or tablets—by conventional coating procedures in a suitable coating pan or in a fluidized bed apparatus using water and/or conventional organic solvents for the coating solution. The material for the separating layer is chosen among the pharmaceutically acceptable, water soluble, inert compounds or polymers used for film-coating applications such as, for instance sugar, polyethylene glycol, polyvinylpyrroline, polyvinyl alcohol, hydroxypropyl cellulose, methylcellulose, hydroxymethyl cellulose, hydroxypropyl methylcellulose, polyvinyl acetal diethylaminoacetate or the like. The thickness of the separating layer is not less than 2 μm, for small spherical pellets preferably not less than 4 μm, for tablets preferably not less than 10 μm.

In the case of tablets another method to apply the coating can be performed by the drycoating technique. First a tablet containing omeprazole is compressed as described above. Around this tablet a layer is compressed using a suitable tableting machine. The outer, separating layer, consists of pharmaceutically acceptable, in water soluble or in water rapidly disintegrating tablet excipients. The separating layer has a thickness of not less than 1 mm. Ordinary plasticizers colorants, pigments, titanium dioxide, talc and other additives may also be included into the separating layer. In case of gelatin capsules the gelatin capsule itself serves as separating layer.

The eneric coating layer is applied on to the sub-coated cores by conventional coating techniques such as, for instance, pan coating or fluidized bed coating using solutions of polymers in water and/or suitable organic solvents or by using latex suspensions of said polymers.

Thus, the special preparation consists of cores containing omeprazole mixed with an alkaline reacting compound or cores containing an alkaline salt of omeprazole optionally mixed with an alkaline reacting compound. The alkaline reacting core material and/or alkaline salt of the active ingredient, omeprazole, enhance the stability of omeprazole. The cores suspended in water forms a solution or a suspension which has a pH, which is higher than that of a solution in which the polymer used for enteric coating is just soluble. The cores are coated with an inert reacting water soluble or in water rapidly disintegrating coating, optionally containing a pH-buffering substance, which separates the alkaline cores from the enteric coating. Without this separating layer the resistance towards gastric juice would be too short and/or the storage stability of the dosage form would be unacceptably short. The sub-coated dosage form is finally coated with an enteric coating rendering the dosage form insoluble in acid media, but rapidly disintegrating/dissolving in neutral to alkaline media such as, for instance the liquids present in the proximal part of the small intestine, the site where dissolution is wanted.

U.S. Pat. Nos. 4,853,230, 5,690,960 (pharmaceutical formulations of omeprazole), U.S. Pat. No. 5,877,192 ((−)-enantiomer of omeprazole), U.S. Pat. No. 5,900,424 (omeprazole magnesium salt form), U.S. Pat. Nos. 6,147,103, 6,166,213, and 6,191,148 (improved process of making omeprazole and the resulting products), U.S. Pat. No. 6,369,085 (S-omeprazole and magnesium salt thereof), U.S. Pat. No. 6,428,810 (pharmaceutical preparation of omeprazole and derivatives thereof), describe various aspects, such as dosage forms, pharmaceutical formulations for specific administration routes (such as oral use, etc.) for omeprazole and related compounds as described above, the entire content of these patents/publications are all incorporated herein be reference.

PREVACID® (lansoprazole) The active ingredient in PREVACID® (lansoprazole) Delayed-Release Capsules, PREVACID® (lansoprazole) for Delayed-Release Oral Suspension, and PREVACID® SoluTab™ Delayed-Release Orally Disintegrating Tablets is a substituted benzimidazole, 2-[[[3-methyl-4-(2,2,2-trifluoroethoxy)-2-pyridyl]methyl]sulfinyl]benzimidazole, a compound that inhibits gastric acid secretion. Its empirical formula is C₁₆H₁₄F₃N₃O₂S with a molecular weight of 369.37. The structural formula is:

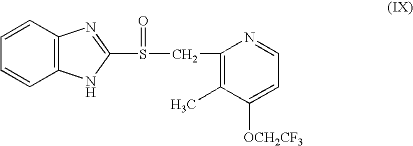

The class of compounds that encompass PREVACID®, and which can be adapted to be used in the instant invention, is described in detail in U.S. Pat. No. 4,255,431 or European Patent application EP 0005129 (supra), the entire contents of which are incorporated herein by reference.

Another related class of compounds with the general formula X below can also be adapted for use in the instant invention:

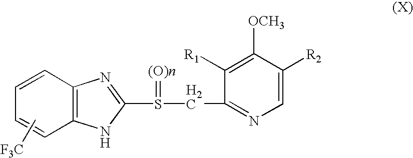

wherein R₁ denotes hydrogen or methyl,

R₂ denotes hydrogen or methyl, and

n denotes the numbers of 0 or 1,

and salts of these compounds.

Among the contemplated salts of compounds of formula X, wherein n denotes 0 (sulfides), are all of the acid-addition salts. The pharmacologically-acceptable salts of the inorganic and organic acids usually employed galenically are notable examples. Pharmacologically-unacceptable salts, which may be obtained initially as process products, for example in the preparation of compounds according to the invention on an industrial scale, are converted into pharmacologically-acceptable salts by conventional processes which are known to the artisan. All acid-addition salts of compounds of formula X which are not pharmacologically acceptable are conventionally converted into either the corresponding free base or into a pharmacologically-acceptable acid-addition salt. Examples of suitable pharmacologically-acceptable salts are water-soluble and water-insoluble acid-addition salts, such as the hydrochloride, hydrobromide, hydroiodide, phosphate, nitrate, sulfate, acetate, citrate, gluconate, benzoate, hibenzate [2-(4-hydroxybenzoyl)benzoate], fendizoate (o-[(2′-hydroxy-4-biphenylyl)carbonyl]benzoate), butyrate, sulfosalicylate, maleate, laurate, malate, fumarate, succinate, oxalate, tartrate, amsonate (4,4′-diaminostilbene-2,2′-disulfonate), embonate [4,4′-methylene-bis-(3-hydroxy-2-naphthoate)], metembonate [4,4′-methylene-bis-(3-methoxy-2-naphthoate)], stearate, tosylate (p-toluenesulfonate), 2-hydroxy-3-naphthoate, 3-hydroxy-2-naphthoate and mesylate (methanesulfonate).

Compounds of formula X, wherein n denotes 1 (sulfoxides) are also convertible into the previously-noted acid-addition salts. These salts, however, do not have the same stability (in aqueous solution) as corresponding salts of the sulfides. On the other hand, the sulfoxides are convertible into their basic salts by reaction with appropriate deprotonization agents, such as inorganic and organic bases. These basic salts are also within the scope of the invention. All basic salts of compounds of formula I which are not pharmacologically acceptable are conventionally converted into either the corresponding free compound or into a pharmacologically-acceptable basic salt.

Illustrative compounds according to the invention are: 4-trifluoromethyl-2-[(4-methoxy-2-pyridylmethyl)thiol]-(1H)-benzimidazole, 4-trifluoromethyl-2-[(4-methoxy-3-methyl-2-pyridylmethyl)thio]-(1H)-benzimidazole, 4-trifluoromethyl-2-[(4-methoxy-5-methyl-2-pyridylmethyl)thio]-(1H)-benzimidazole, 4-trifluoromethyl-2-[(4-methoxy-3,5-dimethyl-2-pyridylmethyl)thio]-(1H)-benzimidazole, 5-trifluoromethyl-2-[(4-methoxy-2-pyridylmethyl)thio]-(1H)-benzimidazole, 5-trifluoromethyl-2-[(4-methoxy-3-methyl-2-pyridylmethyl)thio]-(1H)-benzimidazole, 5-trifluoromethyl-2-[(4-methoxy-5-methyl-2-pyridylmethyl)thio]-(1H)-benzimidazole and 5-trifluoromethyl-2-[(4-methoxy-3,5-dimethyl-2-pyridylmethyl)thio]-(1H)-benzimidazole, 4-trifluoromethyl-2-[(4-methoxy-2-pyridylmethyl)-sulfinyl]-(1H)-benzimidazole, 4-trifluoromethyl-2-[(4-methoxy-3-methyl-2-pyridylmethyl)sulfinyl]-(1H)-benzimidazole, 4-trifluoromethyl-2-[(4-methoxy-5-methyl-2-pyridylmethyl)sulfinyl]-(1H)-benzimidazole, 4-trifluoromethyl-2-[(4-methoxy-3,5-dimethyl-2-pyridylmethyl)sulfinyl]-(1H)-benzimidazole, 5-trifluoromethyl-2-[(4-methoxy-2-pyridylmethyl)sulfinyl]-(1H)-benzimidazole, 5-trifluoromethyl-2-[(4-methoxy-3-methyl-2-pyridylmethyl)sulfinyl]-(1H)-benzimidazole, 5-trifluoromethyl-2-[(4-methoxy-5-methyl-2-pyridylmethyl)sulfinyl]-(1H)-benzimidazole and 5-trifluoromethyl-2-[(4-methoxy-3,5-dimethyl-2-pyridylmethyl)sulfinyl]-(1H)-benzimidazole, and their salts.

Particularly preferred compounds are 5-trifluoromethyl-2-[(4-methoxy-3-methyl-2-pyridylmethyl)thio]-(1H)-benzimidazole, 5-trifluoromethyl-2-[(4-methoxy-3,5-dimethyl-2-pyridylmethyl)sulfinyl]-(1H)-benzimidazole and 5-trifluoromethyl-2-[(4-methoxy-2-pyridylmethyl)sulfinyl]-(1H)-benzimidazole and their pharmacologically-acceptable salts.

Because of tautomerism in the imidazole ring, 4- and 5-substitution in the benzimidazole is identical to 7- and, respectively, 6-substitution.

The sulfoxides according to the invention are optically active compounds. The invention thus includes both the enantiomers and their mixture and racemates. The enantiomers can be separated in a manner which is known to the expert, e.g. by preparation and separation of corresponding diastereoisomers. The enantiomers can also be prepared by asymmetric synthesis [cf. K. K. Andersen, Tetrahedron Lett., 93 (1962)].

These compounds and salts thereof can be made according to the disclosure of U.S. Pat. No. 4,472,409, entire contents of which is incorporated herein by reference.

A preferred class of compounds is described below in formula XI (see U.S. Pat. Nos. 4,628,098, 4,689,333, and 5,013,743, incorporated herein by reference).

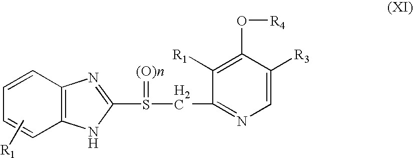

wherein R₁ is hydrogen, methoxy or trifluoromethyl, R₂ and R₃ are independently hydrogen or methyl, R₄ is a C₂₋₅, preferably C₂₋₃ fluorinated alkyl, and n denotes 0 or 1, or their pharmacologically acceptable salts.

In the above formulae, C₂₋₅ fluorinated alkyl groups shown by R₄ are exemplified by 2,2,2-trifluoroethyl, 2,2,3,3,3-pentafluoropropyl, 2,2,3,3-tetrafluoropropyl 1-(trifluoromethyl)-2,2,2-trifluoroethyl, 2,2,3,3,4,4,4-heptafluorobutyl and 2,2,3,3,4,4,5,5-octafluoropentyl.

R₁ may be located at 4- or 5-position, and preferably at 5-position.

In one embodiment, R₁ is hydrogen or trifluoromethyl. In one embodiment, R₃ is hydrogen. In one embodiment, R₁ and R₃ are hydrogen, R₂ is methyl, R₄ is 2,2,2-trifluoroethyl and n is 1.

Examplery compounds of formula XI are:

2-[4-(2,2,2-trifluoroethoxy)-pyrid-2-yl]methylsulfinylbenzimidazole;

2-[3-methyl-4-(2,2,2-trifluoroethoxy)-pyrid-2-yl]methylsulfinylbenzimidazole;

2-[4-(2,2,2-trifluoroethoxy)-5-methyl-pyrid-2-yl]methylsulfinylbenzimidazole;

2-[3-methyl-4-(2,2,2-trifluoroethoxy)-5-methyl-pyrid-2-yl]methylsulfinylbenzimidazole;

2-[4-(2,2,3,3,3-pentafluoropropoxy)-pyrid-2-yl]methylsulfinylbenzimidazole;

2-[4-(2,2,3,3,3-pentafluoropropoxy)-5-methyl-pyrid-2-yl]methylsulfinylbenzimidazole;

2-[4-(2,2,3,3-tetrafluoropropoxy)-pyrid-2-yl]methylsulfinylbenzimidazole;

2-[3-methyl-4-(2,2,3,3,3-pentafluoropropoxy)-pyrid-2-yl]methylsulfinylbenzimidazole;

2-[3-methyl-4-(2,2,3,3-tetrafluoropropoxy)-pyrid-2-yl]methylsulfinylbenzimidazole;

2-[5-methyl-4-(2,2,3,3-tetrafluoropropoxy)-pyrid-2-yl]methylsulfinylbenzimidazole;

2-[3,5-dimethyl-4-(2,2,3,3,3-pentafluoropropoxy)-pyrid-2-yl]methylsulfinylbenzimidazole;

2-[3-methyl-4-(2,2,2-trifluoroethoxy)-pyrid-2-yl]methylsulfinyl-5-trifluoromethylbenzimidazole;

2-[3-methyl-4-(2,2,2-trifluoroethoxy)-pyrid-2-yl]methylsulfinyl-5-methoxybenzimidazole;

2-[4-(2,2,2-trifluoroethoxy)-pyrid-2-yl]-methylsulfinyl-5-methoxybenzimidazole;

2-[4-(2,2,2-trifluoroethoxy)-pyrid-2-yl]-methylthiobenzimidazole;

2-[3-methyl-4-(2,2,2-trifluoroethoxy)-pyrid-2-yl]methylthiobenzimidazole;

2-[5-methyl-4-(2,2,2-trifluoroethoxy)-pyrid-2-yl]methylthiobenzimidazole;

2-[3,5-dimethyl-4-(2,2,2-trifluoroethoxy)-pyrid-2-yl]methylthiobenzimidazole;

2-[4-(2,2,3,3,3-pentafluoropropoxy)-pyrid-2-yl]methylthiobenzimidazole;

2-[5-methyl-4-(2,2,3,3,3-pentafluoropropoxy)-pyrid-2-yl]methylthiobenzimidazole;

2-[4-(2,2,3,3-tetrafluoropropoxy)-pyrid-2-yl]methylthiobenzimidazole;

2-[3-methyl-4-(2,2,3,3-tetrafluoropropoxy)-pyrid-2-yl]methylthiobenzimidazole;

2-[5-methyl-4-(2,2,3,3-tetrafluoropropoxy)-pyrid-2-yl]methylthiobenzimidazole;

2-[3-methyl-4-(2,2,3,3,3-pentafluoropropoxy)-pyrid-2-yl]methylthiobenzimidazole;

2-[3-methyl-4-(2,2,3,3,3-pentafluoropropoxy)-5-methyl-pyrid-2-yl]methylthiobenzimidazole;

2-[3-methyl-4-(2,2,2-trifluoroethoxy)-pyrid-2-yl-methylthio-5-trifluoromethylbenzimidazole;

2-[3-methyl-4-(2,2,2-trifluoroethoxy)-pyrid-2-yl-methylthio-5-methoxybenzimidazole;

2-[4-(2,2,2-trifluoroethoxy)-pyrid-2-yl]methylthio-5-methoxybenzimidazole.

In addition, preferable examples of the hydrocarbon residue in the optionally substituted hydrocarbon shown by R₄ include 1-6 C straight-chain or branched alkyl groups, 2-6 C alkenyl groups and alkynyl groups; the alkyl groups are exemplified by methyl, ethyl, propyl, isopropyl, butyl, 1-methylpropyl, 2-methylpropyl, t-butyl, pentyl, 2-methylbutyl, hexyl, 4-methylpentyl, etc.; the alkenyl groups are exemplified by vinyl, 2-propenyl, 2-butenyl, 3-butenyl, 2-methyl-2-propenyl, 2-pentenyl, 3-pentenyl, 4-pentenyl, 2-methyl-2-butenyl, 3-methyl-2-butenyl, 2-hexenyl, 3-hexenyl, 4-hexenyl, 5-hexenyl, 3-methyl-2-pentenyl, 4-methyl-3-pentenyl, etc.; the alkynyl groups are exemplified by ethinyl, 2-propinyl, 1-methyl-2-propinyl, 2-butynyl, 3-butynyl, 1-methyl-2-butynyl, 2-pentynyl, 3-pentynyl, 4-pentynyl, 2-methyl-3-pentynyl, 2-hexynyl, etc. As the substituents, mention is made of fluorine and 1-3 C alkoxy groups. The number of substituents ranges from 1 to 9, in the case of fluorine, and the number is 1 or 2, in the case of alkoxy groups. Examples of thus substituted compounds include 2,2,2-trifluoroethyl, 2,2,3,3,3-pentafluoropropyl, 2,2,3,3-tetrafluoropropyl, 1,1,1,3,3,3-hexafluoro-2-propyl, 2,2,3,3,4,4,4-heptafluorobutyl, 2,2,3,3,4,4-hexafluorobutyl, 2,2,3,3,4,4,5,5-octaflouropentyl, 2,2,3,3,4,4,5,5,5-nonafluoropentyl, cis-2-fluoro-2-butenyl, 2,2,3,4,4-pentafluoro-3-butenyl, 1,1,1-trifluoro-3-pentyn-2-yl, methoxymethyl, ethoxymethyl, propoxyethyl, 2-methoxypropyl, 3-methoxypropyl, 3-ethoxypropyl, 4-methoxybutyl, trans-3-methoxy-2-propenyl, trans-3-methoxy-2-butenyl, 4-methoxy-2-butynyl, 4methoxy-2-butynyl, etc. Among these, fluorinated 2-6 C straight-chain or branched alkyl groups are especially preferable.

U.S. Pat. Nos. 5,045,321, 5,093,132, 5,433,959, 5,639,478, 5,879,708, 6,017,560, 6,123,962, 6,296,875, and 6,380,234 (incorporatd herein by reference) describes in detail various stabilized pharmaceutical compositions of formula XII, which can be adapted for use in the instant invention.

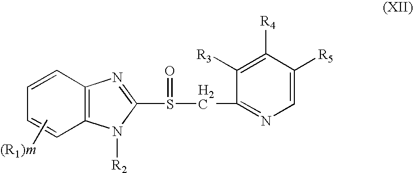

wherein R₁ is hydrogen, alkyl, halogen, cyano, carboxy, carboalkoxy, carboalkoxyalkyl, carbamoyl, carbamoylalkyl, hydroxy, alkoxy, hydroxyalkyl, trifluoromethyl, acyl, carbamoyloxy, nitro, acyloxy, aryl, aryloxy, alkylthio or alkylsulfinyl, R₂ is hydrogen, alkyl, acyl, carboalkoxy, carbamoyl, alkylcarbamoyl, dialkylcarbamoyl, alkylcarbonylmethyl, alkoxycarbonylmethyl or alkylsulfonyl, R₃ and R₅ are the same or different and each is hydrogen, alkyl, alkoxy or alkoxyalkoxy, R₄ is hydrogen, alkyl, alkoxy which may optionally be fluorinated, or alkoxyalkoxy, and m is an integer of 0 through 4.

Referring to R₁ in the above formula, C₁-₇ alkyls may be mentioned as the alkyl represented by R₁; C₁₋₄ alkoxys as the alkoxy moiety of the carboalkoxy; C₁₋₄ alkoxys as the alkoxy moiety of the carboalkoxyalkyl and C₁₋₄ alkyls as the alkyl moiety; C₁₋₄ alkyls as the alkyl moiety of the carbamoylalkyl; C₁₋₅ alkoxys as the alkoxy; C₁₋₇ alkyls as the alkyl moiety o f the hydroxyalkyl; C₁₋₄ alkanoyls as the acyl; phenyl as the aryl; phenyl as the aryl moiety of the aryloxy; C₁₋₆ alkyls as the alkyl moiety of the alkylthio; and C₁₋₆ alkyls as the alkyl moiety of the alkylsulfinyl.

Referring to R₂, C₁₋₅ alkyls may be mentioned as the alkyl represented by R₂; C₁₋₄ alkanoyls as the acyl; C₁₋₄ alkoxys as the alkoxy moiety of the carboalkoxy; C₁₋₄ alkyls as the alkyl moiety of the alkylcarbamoyl; C₁₋₄ alkyls as each of the alkyl moieties of the dialkylcarbamoyl; C₁₋₄ alkyls as the alkyl moiety of the alkylcarbonylmethyl; C₁₋₄ alkoxys as the alkoxy moiety of the alkoxycarbonylmethyl; and C₁₋₄ alkyls as the alkyl moiety of the alkylsulfonyl.

Referrring to R₃, R₄ and R₅, C₁₋₄ alkyls may be mentioned as the alkyl represented by any of them; C₁₋₈ alkoxys as the alkoxy; and C₁₋₄ alkoxys as each of the alkoxy moieties of the alkoxyalkoxy.

Referring to R₄, C₁₋₈ alkoxys may be mentioned as the alkoxy, which may optionally be fluorinated.

Among those compounds of the above forumula XII, (1) the compounds of which R₁ is hydrogen, methoxy or trifluoromethyl, R₂ is hydrogen, R₃ and R₅ are the same or different and each is hydrogen or methyl, R₄ is fluorinated C₂₋₅ alkoxy and m is 1, (2) the compounds of which R₁ is hydrogen, fluorine, methoxy or trifluoromethyl, R₂ is hydrogen, R₃ is hydrogen or methyl, R₄ is C₃-₈ alkoxy, R₅ is hydrogen and m is 1, and (3) the compounds of which R₁ is hydrogen, fluorine, methoxy or trifluoromethyl, R₂ is hydrogen, R₃ is C₁₋₈ alkoxy, R₄ is C₁₋₈ alkoxy which may be fluorinated, R₅ is hydrogen and m is 1.

Detailed mention is now made of the substituents in such novel compounds.

Referring to R₃, the lower alkyl represented thereby is preferably C₁₋₈ lower alkoxy such as methoxy, ethoxy, propoxy, isopropoxy, butoxy, isobutoxy, pentyloxy, hexyloxy, heptyloxy or octyloxy and more preferably C₁₋₄ lower alkoxy.

Referring to R₄, C₁₋₈ lower alkoxys may be mentioned as the lower alkoxy, which may optionally be fluorinated, and preferred examples are as mentioned above for R₃. As the fluorinated lower alkoxy, there may be mentioned, for example, 2,2,2-trifluoroethoxy, 2,2,3,3,3-pentafluoropropoxy, 1-(trifluoromethyl)-2,2,2-trifluoroethoxy, 2,2,3,3-tetrafluoropropoxy, 2,2,3,3,4,4,4-heptafluorobutoxy and 2,2,3,3,4,4,5,5-octafluoropentoxy, and fluorinated C₂₋₄ lower alkoxys are preferred.

The position of R₁ is position 4 or position 5, preferably position 5.

The basic inorganic salt of magnesium and that of calcium, which are to be used in accordance with the invention, are now described.

Said basic inorganic salt of magnesium includes, among others, heavy magnesium carbonate, magnesium carbonate, magnesium oxide, magnesium hydroxide, magnesium metasilicate aluminate, magnesium silicate aluminate, magnesium silicate, magnesium aluminate, synthetic hydrotalcite [Mg₆Al₂(OH)₁₆.CO₃.4H₂)] and aluminum magnesium hydroxide [2.5MgO.Al₂O₃.xH₂O] and said basic inorganic salt of calcium includes, among others, precipitated calcium carbonate and calcium hydroxide. It is only required of such basic inorganic magnesium and calcium salts to show basicity (pH of not less than 7) when they are in the form of a 1% aqueous solution or suspension.

Said basic inorganic magnesium and calcium salts may be used either singly or in combination of two or more species in an amount which may vary depending on the kinds thereof but generally lies within the range of about 0.3-20 parts by weight, preferably about 0.6-7 parts by weight, per part by weight of the benzimidazole compounds.

The composition of the invention may further contain such additives as vehicles (e.g. lactose, corn starch, light silicic anhydride, microcrystalline cellulose, sucrose), binders (e.g. .alpha.-form starch, methylcellulose, carboxymethylcellulose, hydroxypropylcellulose, hydroxypropylmethylcellulose, polyvinylpyrrolidone), disintegrating agents (e.g. carboxymethylcellulose calcium, starch, low substituted hydroxypropylcellulose), surfactants [e.g. Tween 80 (Kao-Atlas), Pluronic F68 (Asahi Denka; polyoxyethylene-polyoxypropylene copolymer], antioxidants (e.g. L-cysteine, sodium sulfite, sodium ascorbate), lubricants (e.g. magnesium stearate, talc), etc.

An examplery pharmaceutical composition is made up into tablets or granules and then coated by a coating agent, which comprises an effective amount of the anti-ulcer compound 2-[[3-methyl-4-(2,2,2-trifluoroethoxy-2-pyridyl]methylsulfinyl] benzimidazole, and at least one of the basic inorganic salts of magnesium and calcium selected from heavy magnesium carbonate, magnesium carbonate, magnesium oxide, magnesium hydroxide, magnesium metasilicate aluminate, magnesium silicate aluminate, magnesium silicate, magnesium aluminate, synthetic hydrotalcite, aluminum magnesium hydroxide, precipitated calcium carbonate and calcium hydroxide; the amount of the basic inorganic salt relative to parts by weight of the benzimidazole compound being about 0.3-20 parts by weight; the benzimidazole compound being in contact with the basic inorganic salt evenly.

PROTONIX® (pantoprazole sodium) The active ingredient in PROTONIX® (pantoprazole sodium) Delayed-Release Tablets is a substituted benzimidazole, sodium 5-(difluoromethoxy)-2-[[(3,4-dimethoxy-2-pyridinyl)methyl]sulfinyl]-1H-benzimidazole sesquihydrate. Its empiricle formula is C₁₆H₁₄F₂N₃NaO₄S×1.5 H₂0, with a molecular weight of 432.4. The structural formula is represented by formula XIII:

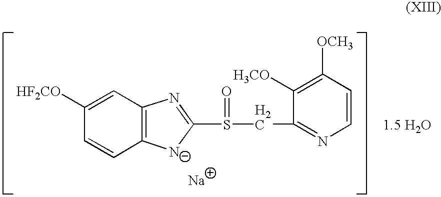

U.S. Pat. No. 4,758,579 (incorporated herein by reference) describes a broader class of dialkoxypyridine compounds that encompasses formula XIII, as shown below in formula XIV below. These compounds can be adapted for use in the instant invention:

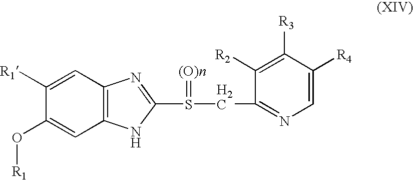

wherein R₁ represents a 1-3C-alkyl radical which is completely or predominantly substituted by fluorine, or a chlorodifluoromethyl radical, and,

R₁′ represents hydrogen (—H), halo, trifluoromethyl, a 1-3C-alkyl radical, or a 1-3C-alkoxy radical which is, optionally, completely or predominantly substituted by fluorine, or,

R₁ and R₁′ together, with inclusion of the oxygen atom to which R₁ is bonded, represent a 1-2C-alkylenedioxy radical which is, optionally, completely or partly substituted by fluorine, or a chlorotrifluoroethylenedioxy radical,

R₃ represents a 1-3C-alkoxy radical,

one of the radicals R₂ and R₄ represents a 1-3C-alkoxy radical and the other represents a hydrogen atom (—H) or a 1-3C-alkyl radical, and,

n represents the number 0 or 1,

and to salts of these compounds.

Examples of 1-3C-alkyl radicals which are completely or predominantly substituted by fluorine are the 1,1,2-trifluoroethyl radical, the perfluoropropyl radical, the perfluoroethyl radical, and in particular, the 1,1,2,2-tetrafluoroethyl radical, the trifluoromethyl radical, the 2,2,2-trifluoroethyl radical and the difluoromethyl radical.

Halogen in the context of the present invention is bromine, chlorine and, in particular, fluorine.

1-3C-alkyl radicals are the propyl, isopropyl, ethyl and, in particular, methyl radical.

1-3C-alkoxy radicals contain, in addition to the oxygen atom, the mentioned 1-3C-alkyl radicals. The methoxy radical is preferred.

1-3C-alkoxy radicals which are completely or predominantly substituted by fluorine contain, in addition to the oxygen atom, the mentioned 1-3C-alkyl radicals which are completely or predominantly substituted by fluorine. Examples include the 1,1,2,2-tetrafluoroethoxy, the trifluoromethoxy, the 2,2,2-trifluoroethoxy and the difluoromethoxy radicals.

Examples of 1-2C-alkylenedioxy radicals which are, optionally, completely or partly substituted by fluorine are the 1,1-difluoroethylenedioxy radical (—O—CF₂—CH₂—O—), the 1,1,2,2;L-tetrafluoroethylenedioxy radical (—O—CF₂—CF₂—O—), the 1,1,2-trifluoroethylenedioxy radical (—O—CF₂—CHF—O—) and, in particular, the difluoromethylenedioxy radical (—O—CF₂—O—), as substituted radicals, and the ethylenedioxy radical and the methylenedioxy radical, as unsubstituted radicals.

Preferred salts of compounds of the formula I in which n denotes the number 0 (sulfides) are all the acid-addition salts. The pharmacologically-acceptable salts of inorganic and organic acids usually employed in galenics are notable examples. Pharmacologically-unacceptable salts which may be obtained initially via industrial-scale processes are converted into pharmacologically-acceptable salts by conventional processes. Examples of suitable pharmacologically-acceptable salts are water-soluble and water-insoluble acid-addition salts, such as the hydrochloride, hydrobromide, hydriodide, phosphate, nitrate, sulfate, acetate, citrate, gluconate, benzoate, hibenzate, fendizoate, butyrate, sulfosalicylate, maleate, laurate, malate, fumarate, succinate, oxalate, tartrate, amsonate, embonate, metembonate, stearate, tosylate, 2-hydroxy-3-naphthoate, 3-hydroxy-2-naphthoate and mesylate.

Preferred salts of compounds of formula XIV in which n denotes 1 (sulfoxides) are basic salts, in particular pharmacologically-acceptable salts with inorganic and organic bases usually employed in pharmacy. Examples of pharmacologically-acceptable basic salts are the sodium, potassium, calcium and aluminum salts.

One embodiment (embodiment a) of the invention comprises compounds of formula XIV wherein R₁′ represents hydrogen (—H), and R₁, R₂, R₃, R₄ and n have the previously-noted meanings; and their salts.

Another embodiment (embodiment b) of the invention comprises compounds of formula XIV wherein R₁′ represents halogen, trifluoromethyl, a 1-3C-alkyl radical or a 1-3C-alkoxy radical which is, optionally, completely or predominantly substituted by fluorine; and R₁, R₂, R₃, R₄ and n have the previously-mentioned meanings; and their salts.

Another embodiment (embodiment c) of the invention comprises compounds of formula XIV wherein R₁ and R₁′ together, including the oxygen atom to which R₁ is bonded, comprise a 1-2C-alkylenedioxy radical, and R₂, R₃, R₄ and n have the aforementioned meanings; and their salts.

Another embodiment (embodiment d) of the invention comprises compounds of formula XIV wherein R₁ and R₁′ together, including the oxygen atom to which R₁ is bonded, comprise a 1-2C-alkylenedioxyradical which is completely or partly substituted by fluorine, and R₂, R₃, R₄ and n have the previously-noted meanings; and their salts.

Another embodiment (embodiment e) of the invention comprises compounds of formula XIV wherein R₁ and R₁′ together, including the oxygen atom to which R₁ is bonded, comprise a chlorotrifluoroethylenedioxy radical, and R₂, R₃, R₄ and n have their previously-ascribed meanings; and their salts.

Preferred compounds of embodiment a are those of formula XIV wherein R₁ represents 1,1,2,2-tetrafluoroethyl, trifluoromethyl, 2,2,2-trifluoroethyl, difluoromethyl or chlorodifluoromethyl, R₁′ represents hydrogen, R₃ represents methoxy, one of the radicals R₂ and R₄ represents methoxy and the other represents hydrogen or methyl, and n represents the number 0 or 1; and the salts of these compounds.

Preferred compounds of embodiment b are those of formula XIV wherein R₁ represents difluoromethyl, R₁′ represents difluoromethoxy or methoxy, R₃ represents methoxy, one of the radicals R₂ and R₄ represent methoxy and the other represents hydrogen or methyl, and n represents the number 0 or 1; and the salts of these compounds.

Preferred compounds of embodiment c are those of formula XIV wherein R₁ and R₁′ together, combined with the oxygen atom to which R₁ is bonded, represent a methylenedioxy or ethylenedioxy radical, R₃ represents methoxy, one of the radicals R₂ and R₄ represents methoxy and the other represents hydrogen or methyl, and n represents the number 0 or 1; and the salts of these compounds.

Preferred compounds of embodiment d are those of formula XIV wherein R₁ and R₁′ together, combined with the oxygen atom to which R₁ is bonded, represent a difluoromethylenedioxy radical or a 1,1,2-trifluoroethylenedioxy radical, R₃ represents methoxy, one of the radicals R₂ and R₄ represents methoxy and the other represents hydrogen or methyl, and n represents the number 0 or 1; and the salts of these compounds.

Preferred compounds of embodiment e are those of formula XIV wherein R₁ and R₁′ together, including the oxygen atom to which R₁ is bonded, represent a chlorotrifluoroethylenedioxy radical, R₃ represents methoxy, one of the radicals R₂ and R₄ represents methoxy and the other represents hydrogen or methyl, and n represents 0 or 1; and the salts of these compounds.

Examples of compounds according to the invention are:

2-[(4,5-dimethoxy-3-methyl-2-pyridyl)methylsulfinyl]-5-trifluoromethoxy-1H-benzimidazole,

2-[(4,5-dimethoxy-3-methyl-2-pyridyl)-methylthio]-5-trifluoromethoxy-1H-benzimidazole,

2-[(4,5-dimethoxy-3-methyl-2-pyridyl)methylsulfinyl]-5-(1,1,2,2-tetrafluoroethoxy)-1H-benzimidazole,

2-[(4,5-dimethoxy-3-methyl-2-pyridyl)methylthio]-5-(1,1,2,2-tetrafluoroethoxy)-1H-benzimidazole,

2-[(4,5-dimethoxy-3-methyl-2-pyridyl)methylsulfinyl]-5-(2,2,2-trifluoroethoxy)-1H-benzimidazole,

2-[(4,5-dimethoxy-3-methyl-2-pyridyl)-methylthio]-5-(2,2,2-trifluoroethoxy)-1H-benzimidazole,

5-difluoromethoxy-2-[(4,5-dimethoxy-3-methyl-2-pyridyl)methylsulfinyl]-1H-benzimidazole,

5-difluoromethoxy-2-[(4,5-dimethoxy-3-methyl-2-pyridyl)methylthio]-1H-benzimidazole,

5-chlorodifluoromethoxy-2-[(4,5-dimethoxy-3-methyl-2-pyridyl)methylsulfinyl]-1H-benzimidazole,

5-chlorodifluoromethoxy-2-[(4,5-dimethoxy-3-methyl-2-pyridyl)methylthio]-1H-benzimidazole,

5,6-bis(difluoromethoxy)-2-[(4,5-dimethoxy-3-methyl-2-pyridyl)methylsulfinyl]-1H-benzimidazole,

5,6-bis(difluoromethoxy)-2-[(4,5-dimethoxy-3-methyl-2-pyridyl)methylthio]-1H-benzimidazole,

5-difluoromethoxy-6-methoxy-2-[(4,5-dimethoxy-3-methyl-2-pyridyl)methylsulfinyl]-1H-benzimidazole,

5-difluoromethoxy-6-methoxy-2-[(4,5-dimethoxy-3-methyl-2-pyridyl)-methylthio]-1H-benzimidazole,

2-[(4,5-dimethoxy-2-pyridyl)methylsulfinyl]-5-trifluoromethoxy-1H-benzimidazole,

2-[(4,5-dimethoxy-2-pyridyl)methylthio]-5-trifluoromethoxy-1H-benzimidazole

2-[(4,5-dimethoxy-2-pyridyl)methylsulfinyl]-5-(1,1,2,2-tetrafluoroethoxy)-1H-benzimidazole,

2-[(4,5-dimethoxy-2-pyridyl)methylthio]-5-(1,1,2,2-tetrafluoroethoxy)-1H-benzimidazole,

2-[(4,5-dimethoxy-2-pyridyl)methylsulfinyl]-5-(2,2,2-trifluoroethoxy)-1H-benzimidazole,

2-[(4,5-dimethoxy-2-pyridyl)methylthio]-5-(2,2,2-trifluoroethoxy)-1H-benzimidazole,

5-difluoromethoxy-2-[(4,5-dimethoxy-2-pyridyl)methylsulfinyl]-1H-benzimidazole,

5-difluoromethoxy-2-[(4,5-dimethoxy-2-pyridyl)methylthio]-1H-benzimidazole,

5-chlorodifluoromethoxy-2-[(4,5-dimethoxy-2-pyridyl)methylsulfinyl]-1H-benzimidazole,

5-chlorodifluoromethoxy-2-[(4,5-dimethoxy-2-pyridyl)methylthio]-1H-benzimidazole,

5,6-bis(difluoromethoxy)-2-[(4,5-dimethoxy-2-pyridyl)methylsulfinyl]-1H-benzimidazole,

5,6-bis(difluoromethoxy)-2-[(4,5-dimethoxy-2-pyridyl)methylthio]-1H-benzimidazole,

5-difluoromethoxy-6-methoxy-2-[(4,5-dimethoxy-2-pyridyl)methylsulfinyl]-1H-benzimidazole,

5-difluoromethoxy-6-methoxy-2-[4,5-dimethoxy-2-pyridyl)methylthio]-1H-benzimidazole,

2-[(3,4-dimethoxy-5-methyl-2-pyridyl)methylsulfinyl]-5-trifluoromethoxy-1H-benzimidazole,

2-[(3,4-dimethoxy-5-methyl-2-pyridyl)methylthio]-5-trifluoromethoxy-1H-benzimidazole,

2,[(3,4-dimethoxy-5-methyl-2-pyridyl)methylsulfinyl]-5-(1,1,2,2-tetrafluoroethoxy)-1H-benzimidazole,

2-[(3,4-dimethoxy-5-methyl-2-pyridyl)methylthio]-5-(1,1,2,2-tetrafluoro-ethoxy)-1H-benzimidazole,

2-[(3,4-dimethoxy-5-methyl-2-pyridyl)methylsulfinyl]-5-(2,2,2-trifluoroethoxy)-1H-benzimidazole,

2-[(3,4-dimethoxy-5-methyl-2-pyridyl)-methylthio]-5-(2,2,2-trifluoroethoxy)-1H-benzimidazole,

5-difluoromethoxy-2-[(3,4-dimethoxy-5-methyl-2-pyridyl)methylsulfinyl]-1H-benzimidazole,

5-difluoromethoxy-2-[(3,4-dimethoxy-5-methyl-2-pyridyl)methylthio]-1H-benzimidazole,

5-chlorodifluoromethoxy-2-[(3,4-dimethoxy-5-methyl-2-pyridyl)methylsulfinyl]-1H-benzimidazole,

5-chlorodifluoromethoxy-2-[(3,4-dimethoxy-5-methyl-2-pyridyl)methylthio]-1H-benzimidazole,

5,6-bis(difluoromethoxy)-2-[(3,4-dimethoxy-5-methyl-2-pyridyl)methylsulfinyl]-1H-benzimidazole,

5,6-bis(difluoromethoxy)-2-[(3,4-dimethoxy-5-methyl-2-pyridyl)methylthio]-1H-benzimidazole,

5-difluoromethoxy-6-methoxy-2-[(3,4-dimethoxy-5-methyl-2-pyridyl)methylsulfinyl]-1H-benzimidazole,

5-difluoromethoxy-6-methoxy-2-[(3,4-dimethoxy-5-methyl-2-pyridyl)methylthio]-1H-benzimidazole,

2-[(3,4-dimethoxy-2-pyridyl)methylsulfinyl]-5-trifluoromethoxy-1H-benzimidazole,

2-[(3,4-dimethoxy-2-pyridyl)methylthio]-5-trifluoromethoxy-1H-benzimidazole

2-[(3,4-dimethoxy-2-pyridyl)methylsulfinyl]-5-(1,1,2,2-tetrafluoroethoxy)-1H-benzimidazole,

2-[(3,4-dimethoxy-2-pyridyl)methylthio]-5-(1,1,2,2-tetrafluoroethoxy)-1H-benzimidazole,

2-[(3,4-dimethoxy-2-pyridyl)methylsulfinyl]-5-(2,2,2-trifluoroethoxy)-1H-benzimidazole,

2-[(3,4-dimethoxy-2-pyridyl)methylthio]-5-(2,2,2-trifluoroethoxy)-1H-benzimidazole,

5-difluoromethoxy-2-[(3,4-dimethoxy-2-pyridyl)methylsulfinyl]-1H-benzimidazole,

5-difluoromethoxy-2-[(3,4-dimethoxy-2-pyridyl)methylthio]-1H-benzimidazole,

5-chlorodifluoromethoxy-2-[(3,4-dimethoxy-2-pyridyl)methylsulfinyl]-1H-benzimidazole,

5-chlorodifluoromethoxy-2-[(3,4-dimethoxy-2-pyridyl)methylthio]-1H-benzimidazole,

5,6-bis(difluoromethoxy)-2-[(3,4-dimethoxy-2-pyridyl)methylsulfinyl]-1H-benzimidazole,

5,6-bis(difluoromethoxy)-2-[(3,4-dimethoxy-2-pyridyl)methylthio]-1H-benzimidazole,

5-difluoromethoxy-6-methoxy-2-[(3,4-dimethoxy-2-pyridyl)methylsulfinyl]-1H-benzimidazole,

5-difluoromethoxy-6-methoxy-2-[(3,4-dimethoxy-2-pyridyl)methylthio]-1H-benzimidazole,

2,2-difluoro-6-[(4,5-dimethoxy-2-pyridyl)methylsulfinyl]-5H-[1,3]-dioxolo-[4,5-f]benzimidazole,

2,2-difluoro-6-[(4,5-dimethoxy-2-pyridyl)methylthio]-5H-[1,3]-dioxolo-[4,5-f]benzimidazole,

2,2-difluoro-6-[(3-methyl-4,5-dimethoxy-2-pyridyl)methylthio]-5H-[1,3]-dioxolo[4,5-f]benzimidazole,

2,2-difluoro-6-[(3-methyl-4,5-dimethoxy-2-pyridyl)methylsulfinyl]-5H-[1,3]-dioxolo[4,5-f]benzimidazole,

6-[(4,5-diethoxy-3-methyl-2-pyridyl)methylthio]-2,2-difluoro-5H-[1,3]-dioxolo[4,5-f]benzimidazole,

6-[(4,5-diethoxy-3-methyl-2-pyridyl)methylsulfinyl]-2,2-difluoro-5H-[1,3]-dioxolo[4,5-f]benzimidazole,

6,6,7-trifluoro-6,7-dihydro-2-[(4,5-dimethoxy-3-methyl-2-pyridyl)methylthio-1H-[1,4]-dioxino[2,3-f]benzimidazole,

6,6,7-trifluoro-6,7-dihydro-2-[(4,5-dimethoxy-3-methyl-2-pyridyl)methylsulfinyl]-1H-[1,4]-dioxino[2,3-f]benzimidazole,

6,6,7-trifluoro-6,7-dihydro-2-[(4,5-dimethoxy-2-pyridyl)methylthio]-1H-[1,4]-dioxino-[2,3-f]benzimidazole,

6,6,7-trifluoro-6,7-dihydro-2-[(4,5-dimethoxy-2-pyridyl)methylsulfinyl]-1H-[1,4]-dioxino[2,3-f]benzimidazole,

2-[(4,5-diethoxy-2-pyridyl)methylthio]6,6,7-trifluoro-6,7-dihydro-1H-[1,4]-dioxino[2,3-f]benzimidazole,

2-[(4,5-diethoxy-2-pyridyl)methylsulfinyl]-6,6,7-trifluoro-6,7-dihydro-1H-[1,4]-dioxino[2,3-f]benzimidazole,

2-[(4,5-diethoxy-3-methyl-2-pyridyl)methylthio]-6,6,7-trifluoro-6,7-dihydro-1H-[1,4]-dioxino[2,3-f]benzimidazole,

2-[(4,5-diethoxy-3-methyl-2-pyridyl)methylsulfinyl]-6,6,7-trifluoro-6,7-dihydro-1H-[1,4]-dioxino[2,3-f]benzimidazole,

6,6-difluoro-6,7-dihydro-2-[(4,5-dimethoxy-2-pyridyl)methylthio]-1H-[1,4]-dioxino[2,3-f]benzimidazole,

6,6-difluoro-6,7-dihydro-2-[(4,5-dimethoxy-2-pyridyl)methylsulfinyl]-1H-[1,4]-dioxino[2,3-f]benzimidazole,

6,6-difluoro-6,7-dihydro-2-[(4,5-dimethoxy-3-methyl-2-pyridyl)methylthio]-1H-[1,4]-dioxino[2,3-f]benzimidazole,

6,6-difluoro-6,7-dihydro-2-[(4,5-dimethoxy-3-methyl-2-pyridyl)methylsulfinyl]-1H-[1,4]-dioxino[2,3-f]benzimidazole,

6,6,7,7-tetrafluoro-6,7-dihydro-2-[(4,5-dimethoxy-2-pyridyl)methylthio]-1H-[1,4]-dioxino[2,3-f]benzimidazole,

6,6,7,7-tetrafluoro-6,7-dihydro-2-[(4,5-dimethoxy-2-pyridyl)methylsulfinyl]-1H-[1,4]-dioxino[2,3-f]benzimidazole,

6,6,7,7-tetrafluoro-6,7-dihydro-2-[(4,5-dimethoxy-3-methyl-2-pyridyl)methylthio]-1H-[1,4]-dioxino[2,3-f]benzimidazole,

6,6,7,7-tetrafluoro-6,7-dihydro-2-[(4,5-dimethoxy-3-methyl-2-pyridyl)methylsulfinyl]-1H-[1,4]-dioxine[2,3-f]benzimidazole,

6-chloro-6,7,7-trifluoro-6,7-dihydro-2-[(4,5-dimethoxy-3-methyl-2-pyridyl)methylsulfinyl]-1H-[1,4]dioxino[2,3-f]benzimidazole,

6-chloro-6,7,7-trifluoro-6,7-dihydro-2-[(4,5-dimethoxy-3-methyl-2-pyridyl)methylthio]-1H-[1,4]-dioxino[2,3-f]benzimidazole,

6-chloro-6,7,7-trifluoro-6,7-dihydro-2-[(4,5-dimethoxy-2-pyridyl)methylsulfinyl]-1H-[1,4]-dioxino[2,3-f]-benzimidazole,

6-chloro-6,7,7-trifluoro-6,7-dihydro-2-[(4,5-dimethoxy-2-pyridyl)methylthio]-1H-[1,4]-dioxino[2,3-f]benzimidazole,

2,2-difluoro-6-[(3,4-dimethoxy-2-pyridyl)methylsulfinyl]-5H-[1,3]-dioxolo[4,5-f]benzimidazole,

2,2-difluoro-6-[(3,4-dimethoxy-2-pyridyl)methylthio]-5H-[1,3]-dioxolo-[4,5-f]benzimidazole,

2,2-difluoro-6-[(3,4-dimethoxy-5-methyl-2-pyridyl)methylthio]-5H-[1,3]-dioxolo[4,5-f]benzimidazole,

2,2-difluoro-6-[(3,4-dimethoxy-5-methyl-2-pyridyl)methylsulfinyl]-5H-[1,3]-dioxolo[4,5-f]benzimidazole,

6-[(3,4-diethoxy-5-methyl-2-pyridyl)methylthio]-2,2-difluoro-5H-[1,3]-dioxolo[4,5-f]benzimidazole,

6-[(3,4-diethoxy-5-methyl-2-pyridyl)methylsulfinyl]-2,2-difluoro-5H-[1,3]-dioxolo[4,5-f]benzimidazole,

6,6,7-trifluoro-6,7-dihydro-2-[(3,4-dimethoxy-5-methyl-2-pyridyl)methylthio]-1H-[1,4]-dioxino[2,3-f]benzimidazole,

6,6,7-trifluoro-6,7-dihydro-2-[(3,4-dimethoxy-5-methyl-2-pyridyl)methylsulfinyl]-1H-[1,4]-dioxino[2,3-f]benzimidazole,

6,6,7-trifluoro-6,7-dihydro-2-[(3,4-dimethoxy-2-pyridyl)methylthio]-1H-[1,4]-dioxino[2,3-f]benzimidazole,

6,6,7-trifluoro-6,7-dihydro-2-[(3,4-dimethoxy-2-pyridyl)methylsulfinyl]-1H-[1,4]-dioxino[2,3-f]benzimidazole,

2-[(3,4-diethoxy-2-pyridyl)methylthio]-6,6,7-trifluoro-6,7-dihydro-1H-[1,4]-dioxino[2,3-f]benzimidazole,

2-[(3,4-diethoxy-2-pyridyl)methylsulfinyl]-6,6,7-trifluoro-6,7-dihydro-1H-[1,4]-dioxino[2,3-f]benzimidazole,

2-[(3,4-diethoxy-5-methyl-2-pyridyl)methylthio]-6,6,7-trifluoro-6,7-dihydro-1H-[1,4]-dioxino[2,3-f]benzimidazole,

2-[(3,4-diethoxy-5-methyl-2-pyridyl)methylsulfinyl]-6,6,7-trifluoro-6,7-dihydro-1H-[1,4]-dioxino[2,3-f]benzimidazole,

6,6-difluoro-6,7-dihydro-2-[(3,4-dimethoxy-2-pyridyl)methylthio]-1H-[1,4]-dioxino[2,3-f]benzimidazole,

6,6-difluoro-6,7-dihydro-2-[(3,4-dimethoxy-2-pyridyl)methylsulfinyl]-1H-[1,4]-dioxino[2,3-f]benzimidazole,

6,6-difluoro-6,7-dihydro-2-[(3,4-dimethoxy-5-methyl-2-pyridyl)methylthio]-1H-[1,4]-dioxino[2,3-f]benzimidazole,

6,6-difluoro-6,7-dihydro-2-[(3,4-dimethoxy-5-methyl-2-pyridyl)methylsulfinyl]-1H-[1,4]-dioxino[2,3-f]benzimidazole,

6,6,7,7-tetrafluoro-6,7-dihydro-2-[(3,4-dimethoxy-2-pyridyl)methylthio]-1H-[1,4]-dioxino[2,3-f]benzimidazole,

6,6,7,7-tetrafluoro-6,7-dihydro-2-[(3,4-dimethoxy-2-pyridyl)methylsulfinyl]-1H-[1,4]-dioxino[2,3-f]benzimidazole,

6,6,7,7-tetrafluoro-6,7-dihydro-2-[(3,4-dimethoxy-5-methyl-2-pyridyl)methylthio]-1H-[1,4]-dioxino[2,3-f]benzimidazole,

6,6,7,7-tetrafluoro-6,7-dihydro-2-[(3,4-dimethoxy-5-methyl-2-pyridyl)methylsulfinyl]-1H-[1,4]-dioxino[2,3-f]benzimidazole,

6-chloro-6,7,7-trifluoro-6,7-dihydro-2-[(3,4-dimethoxy-5-methyl-2-pyridyl)methylsulfinyl]-1H-[1,4]-dioxino[2,3-f]benzimidazole,

6-chloro-6,7,7-trifluoro-6,7-dihydro-2-[(3,4-dimethoxy-5-methyl-2-pyridyl)methylthio]-1H-[1,4]-dioxino[2,3-f]benzimidazole,

6-chloro-6,7,7-trifluoro-6,7-dihydro-2-[(3,4-dimethoxy-2-pyridyl)methylsulfinyl]-1H-[1,4]-dioxino[2,3-f]benzimidazole,

6-chloro-6,7,7-trifluoro-6,7-dihydro-2-[(3,4-dimethoxy-2-pyridyl)methylthio]-1H-[1,4]-dioxino[2,3-f]benzimidazole,

6-[(4,5-dimethoxy-3-methyl-2-pyridyl)-methylthio]-5H-[1,3]dioxolo[4,5-f]benzimidazole,

6-[(4,5-dimethoxy-3-methyl-2-pyridyl)-methylsulfinyl]-5H-[1,3]-dioxolo[4,5-f]benzimidazole,

6-[(4,5-dimethoxy-2-pyridyl)methylthio]-5H-[1,3]dioxolo[4,5-d]benzimidazole

6-[(4,5-dimethoxy-2-pyridyl)methylsulfinyl]-5H-[1,3]-dioxolo[4,5-f]benzimidazole,

6-[(3,4-dimethoxy-2-pyridyl)-methylthio]-5H-[1,3]-dioxolo[4,5-f]benzimidazole,

6-[(3,4-dimethoxy-2-pyridyl)methylsulfinyl]-5H-[1,3]-dioxolo[4,5-f]benzimidazole.

6-[(3,4-dimethoxy-5-methyl-2-pyridyl)methylthio]-5H-[1,3]-dioxolo[4,5-f]-benzimidazole,

6-[(3,4-dimethoxy-5-methyl-2-pyridyl)methylsulfinyl]-5H-[1,3]-dioxolo[4,5-f]benzimidazole,

6,7-dihydro-2-[(4,5-dimethoxy-3-methyl-2-pyridyl)methylthio]-1H-[1,4]-dioxino[2,3-f]benzimidazole,

6,7-dihydro-2-[(4,5-dimethoxy-3-methyl-2-pyridyl)methylsulfinyl]-1H-[1,4]-dioxino[2,3-f]benzimidazole,

6,7-dihydro-2-[(3,4-dimethoxy-5-methyl-2-pyridyl)methylthio]-1H-[1,4]-dioxino[2,3-f]benzimidazole,

6,7-dihydro-2-[(3,4-dimethoxy-5-methyl-2-pyridyl)methylsulfinyl]-1H-[1,4]-dioxino[2,3-f]benzimidazole,

6,7-dihydro-2-[(3,4-dimethoxy-2-pyridyl)methylthio]-1H-[1,4]-dioxino[2,3-f]benzimidazole and

6,7-dihydro-2-[(4,5-dimethoxy-2-pyridyl)methylsulfinyl]-1H-[1,4]-dioxino[2,3-f]benzimidazole,

and salts of these compounds.

Due to the tautomerism in the imidazole ring, 5-substitution in the benzimidazole is identical to 6-substitution. Accordingly, in the compounds in which R₁ and R₁′ together, with inclusion of the oxygen atom to which R₁ is bonded, represent a substituted ethylenedioxy radical, the 6-position in the [1,4]-dioxino[2,3-f]benzimidazole part is identical to the 7-position.

U.S. Pat. No. 5,997,903 describes oral presentation forms for pantoprazole, which consist of a core, an intermediate layer and an outer layer which is resistant to gastric juice. The entire content of the patent is incorporated herein by reference.

ACIPHEX® (rabeprazole sodium or pariprazole) The active ingredient in ACIPHEX® Delayed-Release Tablets is rabeprazole sodium, a substituted benzimidazole that inhibits gastric acid secretion. Rabeprazole sodium is known chemically as 2-[[[4-(3-methoxypropoxy)-3-methyl-2-pyridinyl]-methyl]sulfinyl]-1H-benzimidazole sodium salt. It has an empirical formula of C₁₈H₂₀N₃NaO₃S and a molecular weight of 381.43. Rabeprazole sodium is a white to slightly yellowish-white solid. It is very soluble in water and methanol, freely soluble in ethanol, chloroform and ethyl acetate and insoluble in ether and n-hexane. The stability of rabeprazole sodium is a function of pH; it is rapidly degraded in acid media, and is more stable under alkaline conditions. The structural formula XV is:

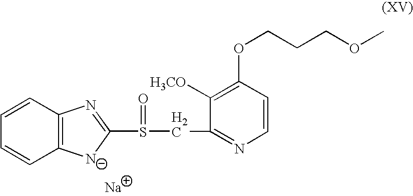

ACIPHEX® is encompassed by a class of compounds represented by formula XVI, which is more potent in anti-ulcer activity than Omeprazole, and is expected to be more potent than Omeprazole for use in the instant invention.

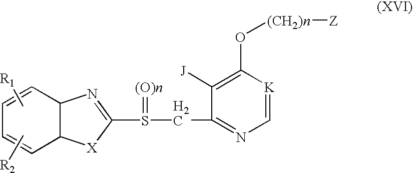

where R₁ and R₂ may be the same or different, each being a hydrogen atom, a lower alkyl, lower alkoxy, halogenated lower alkyl, lower alkoxycarbonyl or carboxyl group or a halogen atoms;

X is a group represented by the formula:

—O—, —S— or

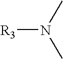

(in which R₃ stands for a hydrogen atom or a lower alkyl, phenyl, benzyl or lower alkoxycarbonyl group); and

Z represents:

1. a group of the formula:

—O—(CH₂)p—O—R₄

where p is an integer of 1 to 3, and R₄ is hydrogen atom or a lower alkyl, aryl or aralkyl group,

2. a group of the general formula:

—O—(CH₂)q—R₅

where q is an integer of 1 to 3, and R₅ is a halogen atom or an alkoxycarbonyl, aryl or heteroaryl group,

3. a group of the general formula:

—O—(CH₂)r—O—(CH2)s—O—R₆

where r and s each independently are an integer of 1 to 5, and R₆ is a hydrogen atom or a lower alkyl group,

4. a group of the formula:

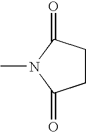

5. a group of the formula:

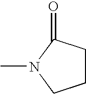

6. a group of the formula:

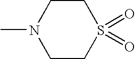

7. a group of the general formula:

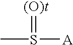

where t is an integer of 0 to 2, and A is a group of the general formula:

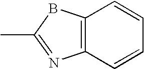

(where B is a group represented by the formula: —NH—, —O— or —S—), a lower alkyl, alkoxycarbonylmethyl, pyridyl or furyl group or a group of the general formula:

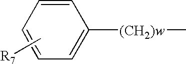

(wherein R₇ stands for a hydrogen atom, a lower alkyl or lower alkoxy group or a halogen atom, and w stands for an integer of 0 or 1).

8. a group of the general formula:

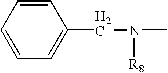

where R₈ is an acetoxy or lower alkyl group, or

9. a group of the general formula:

—O—R₉

where R₉ is a hydrogen atom or a lower alkyl or aryl group;

n is an integer of 0 to 2; m is an integer of 2 to 10, and,

J and K, which may be the same of different from each other, each stand for a hydrogen atom or a lower alkyl group, with the proviso that when Z is a group falling under the above category (9) R₉ is a lower alkyl group and m stands for an integer of 3 to 10,

and pharmaceutically acceptable salts thereof.

Also disclosed are pharmaceutical compositions containing these compounds as the active ingredient(s) for preventing or treating snoring in mammals, including humans, using these pharmaceutical compositions.

In the definition of the compounds of general formula XVI given above, the lower alkyl group defined above with respect to R₁, R₂, R₃, R₄, R₆, A, R₇, R₈, J and K in the compound of the present invention may be a straight-chain or branched alkyl groups having 1 to 6 carbon atoms. Examples include methyl, ethyl, n-propyl, n-butyl, isopropyl, isobutyl, 1-methylpropyl, tert-butyl, n-pentyl, 1-ethylpropyl, isoamyl and n-hexyl groups, among which methyl and ethyl groups are most preferred.

The lower alkoxy group and the lower alkoxy moiety of the lower alkoxycarbonyl group defined above with respect to R₁ and R₂ may be an alkoxy group derived form the above lower alkyl group. Methoxy and ethoxy groups are most preferred.

The halogen atom defined above includes chlorine, bromine, iodine or fluorine. The aryl group defined above with respect to R₄ and R₅ may be phenyl, tolyl, xylyl, naphthyl or the like which may be substituted with a lower alkoxy or hydroxyl group, a halogen atom or the like.

Examples of the arylalkyl defined above with respect to R₄ include benzyl and phenethyl groups.

Examples of the heteroaryl group defined above with respect to R₅ include pyridyl and furyl groups.

In the definition of Z in general formula XVI, groups 1, 2, 3, 4, 5 and 9 are preferred; group 9 is the most preferred. As for R₁ and R₂, hydrogens for both and then a combination of a lower alkyl, inter alia methyl, for R₁ and hydrogen for R₂ are preferred. X is preferably —NR₃ where R₃ is hydrogen. A preferred value for n is 1. The preferred substituents for J and K are both hydrogen or where J is lower alkyl, inter alia methyl, and K is hydrogen, or when J is hydrogen K is lower alkyl, inter alia methyl. Thus, J or K are independently preferably hydrogen or methyl, most preferably J is methyl and K is hydrogen.

A first preferred class of compounds falling within the compounds of general formula XVI are represented by the following formula XVII:

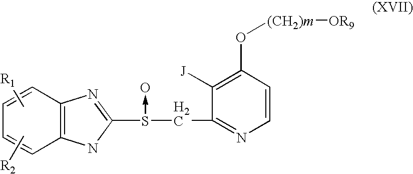

(where R₁, R₂, J, m and R₉ have the same meanings as defined above). In formula XVII, the preferred R₁ and R₂ substituents are both hydrogen, or R₁ is 5-lower alkoxy, 5-lower alkyl or 5-halogenated lower alkyl and R₂ is hydrogen. The preferred substituent for J is hydrogen or methyl; the preferred value for m is in the range of 3 to 10, the most preferred being 3; and the preferred R₉ substituent is lower alkyl, inter alia methyl, or aryl. Among these possibilities for the compounds of formula A the preferred combination is when R₁ and R₂ are both hydrogen, J is methyl, m is 3 and R₉ is methyl.

A second group of preferred compounds are combinations of the above substituents where both R¹ and R² are hydrogen, J is hydrogen, m is 3 and R₉ is methyl.

A third group of preferred compounds falling within formula XVII is when both R₁ and R₂ are hydrogen, J is methyl, m is 2 and R₉ is benzyl.

A second class of compounds falling within general formula XVI are represented by the following formula XVIII:

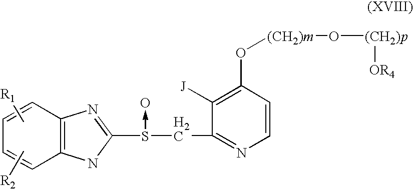

(where R₁, R₂, J, p, m and R₄ have the same meanings as given above). In formula XVIII, the preferred substituents for R₁ and R₂ are both hydrogen; or when R₁ is 5-lower alkoxy, 5-lower alkyl or 5-halogenated lower alkyl, R₂ is hydrogen. The preferred value of m is 2 or 3; the preferred value for p is 2 or 3; and the preferred substituent for R₄ is methyl or benzyl. Of the above possibilities for formula (B), the most preferred combination is where R₁ is 5-methyl, R₂ is hydrogen, J is methyl, m is 2, p is 2 and R₄ is methyl.

Examples of the pharmaceutically acceptable salt include salts with inorganic acids, such as hydrochloride, hydrobromide, sulfate and phosphate; those with organic acids, such as acetate, maleate, tartrate, methanesulfonate, benzenesulfonate, and toluenesulfonate; and those with amino acids such as arginine, aspartic acid and glutamic acid.

Some of the compounds according to the present invention can form a salt with a metal such as Na, K, Ca or Mg. These metal salts are also included among the pharmaceutically acceptable salts of the present invention. For example, compounds represented by the general formula (I), wherein X is a group of —N—R₃, and R₃ is a hydrogen atom, or compounds represented by the general formula XVI, wherein Z is a group falling under category 7 and B is a group of —NH—, can be present as a metal salt.

A most preferable, acid-unstable compound is sodium salt of 2((4-(3-methoxypropoxy)-3-methylpyridin-2-yl)methylsulfinyl)-1H-benzimidazol.

Although the compounds of the present invention may also be present as a hydrate or as a stereoisomer, it is a matter of course that these hydrates and stereoisomers are also included in the scope of the present invention.

These compounds (formulas XVI, XVII, and XVIII) are described in detail in European patent application EP 0268956 and U.S. Pat. No. 5,045,552, the entire contents of which are incorporated herein by reference.

U.S. Pat. No. 6,150,380 describes a novel crystalline form (A) of omeprazole, which is different than the previously described crystalline form (B) of omeprazole. The entire contents incorporated herein by reference.

The preparations may also include an anti-histamine, a decongestant, and/or an anti-inflammatory agent.

Dosing information for each of these known pharmaceutical compositions is described, for example, in Physician's Desk Reference (Medical Economics Co., Inc., Montvale, N.J., 551st ed., 1997). Dosing information for using each of these known pharmaceutical compositions in the methods of the invention are the same as the dosing information for the use of each of these known pharmaceutical compositions for inhibiting gastric secretion, for example in the treatment of GERD. Methods of adapting the dosing information to individual human patients are within the ordinary level of skill in the art.

As used herein, an “effective amount” of an inhibitor of gastric secretion is an amount which, when administered to a human, causes a significant decrease in the amount of gastric juice and acid which is secreted by the human, the significant decrease being a decrease of at least 10%, and preferably 25%, 50%, 75%, or more than 75%.

To determine the effectiveness of the subject compositions in treating snoring, there are a variety of ways to quantify and measuring snoring. Those include:

Intensity—(loudness, frequency, and duration) correlate with the degree of obstruction

Sleep sonography—measures and records the sounds of snoring

In preferred embodiments, the subject compositions include appropriate doses to produce clinically significant effects in patients.

To determine the effectiveness of the subject compositions in treating sleep apnea, there are a variety of ways to quantify and measuring snoring. Those include:

Visual observation of sleep, to detect labored breathing, with long pauses, followed by arousal from sleep.

Pulse oximetry, measuring of the amount of oxygen in the blood and the pulse rate. Multiple dips in oxygen level and peaks in pulse rate are found in people with sleep apnoea.

Polysonmography, which involves many measurements of sleep, including eye movements and chin tone to define sleep stages, flow of air through the nose and mouth, movement of the chest wall, oxygen levels in the blood, and ECG (electrocardiography) to measure any serious abnormal heart rhythms.

In the treatment of sleep apnea, the subject method preferably produces a clinically significant improvement in one or more of these tests, and/or a decrease in a patient's Epworth sleepiness score (such as into the range of 0-10).

### EXAMPLES

### Example 1

The effectiveness of Prevacid (Lansoprazole) is demonstrated in the following case study of a 55-year-old nocturnal breathing obstructed white female suffering with gastroesophageal reflux disease (GERD):

A 55-year-old white female, 5 feet 3 inches tall and moderately overweight reported a 15-20 year history of progressively increasing snoring. The intensity of her snoring was well documented by her husband, who experienced disturbed sleep as a result of his wife's snoring, occasionally requiring him to waken the patient in order for him to obtain relief. The patient gave no history of naso-pharyngeal structural abnormalities or surgery (other than third molar extraction) and had experienced no breathing disorder.

The patient complained to her physician of a chronic scratchiness in her throat, thought to be a result of gastroesophageal reflux disease (GERD). To treat the GERD symptoms, the patient was placed on Prevacid (Lansoprazole) 15 mg once daily. After beginning treatment, the throat scratchiness declined promptly. In addition, her husband made an unexpected observation that there was also a gradual decline in the intensity of the patient's snoring. This moderate to marked benefit was sustained for the duration of dosing, which lasted approximately 12 months. Dosing was then interrupted for a period of several weeks with the subsequent resumption of noticeable snoring intensity.

Therapy with a different inhibitor of gastric acid secretion, Zantac (ranitidine) 75 mg once daily was then begun and the snoring intensity was gradually reversed over the next few weeks. Throughout the subsequent year the beneficial effect on snoring was maintained with gastric acid inhibitors including Tagamet (Cimetidine) and Pepcid AC (Famotidine).

### Example 2

An open label study was performed on 8 outpatients with significant snoring. The patients were treated with Prevacid 30 mg. for 30-90 days. The entry criteria were snoring with or without sleep apnea, documented by history, physical exam, and independent sleep lab studies. None of the patients enrolled in the study had had a diagnosis of gastrointestinal reflux (GERD). At baseline patients had all been placed on conservative nasal regimen with no improvement in their symptoms. Each had a global assessment of breathing and/or sleep related disorders and a sleep lab diagnostic evaluation. At the end of treatment changes in global and spousal ratings of snoring were made along with investigator observations.

The demographics of the study group were eight males no females; a mean age of 50.6 years with a range of 32-70; a mean weight of 206 lbs with a range of 160-260; and there were 6 white, 1 black, and 1 Asian in the group. At baseline the investigator made the diagnosis of GERD in 4 patients. Spousal ratings of snoring were 3 moderate and 5 severe; 4 had moderate or severe sleep apnea; and all 8 had daytime sleepiness.

The duration of treatment ranged from 35 to 90 days with a mean of 61 days. At conclusion of the study, 7 of 8 patients had moderate or marked improvement in snoring. (The one patient who showed no improvement in snoring had already been receiving antacid pharmacologic treatment). 8 of 8 patients had marked improvement in their sleep with less waking and less daytime fatigue.

All publications, patents and patent applications are herein incorporated by reference in their entirety to the same extent as if each individual publication, patent or patent application was specifically and individually indicated to be incorporated by reference in its entirety.

Equivalents

Those skilled in the art will recognize, or be able to ascertain using no more than routine experimentation, many equivalents to the specific embodiments of the invention described herein. Such equivalents are intended to be encompassed by the following claims.

1. A method for treating snoring, sleep apnea and other forms of sleep disordered breathing, comprising administering to a patient an agent for treating symptoms of hyper-acidity or gastro-intestinal reflux disease (GERD).

2. A method for treating respiratory impairment while awake, comprising the administering to a patient an agent for treating symptoms of hyper-acidity or gastro-intestinal reflux disease (GERD).

3. A packaged pharmaceutical comprising:
(i) a pharmaceutical preparation of an agent for treating symptoms of hyper-acidity or gastro-intestinal reflux disease and a pharmaceutically acceptable excipient, which preparation includes an amount of said agent(s) sufficient to reduce the symptoms or frequency of occurrence of sleep disordered breathing in a patient; and
(ii) instructions for use of the preparation by a human patient for reducing the symptoms or frequency of occurrence of sleep disordered breathing.

4. Use of an agent for treating symptoms of hyper-acidity or gastro-intestinal reflux disease in the manufacture of a medicament for reducing the symptoms or frequency of occurrence of sleep disordered breathing in a human patient.

5. The method of claim 1 or 2, the packaged pharmaceutical of claim 3, or the use of claim 4, wherein said inhibitor is an H2 histamine receptor antagonist, an inhibitor of H⁺, K⁺ ATPase, a proton pump inhibitor, a Bismuth compound, an antacid, a synthetic analog of somatostatin, an antiemetic agent, a sucralfate, prostaglandin analog, a muscarinic cholinergic antagonist, a D2 antagonist, a chenodeoxycholic acid, an ursodeoxycholic acid, or a pancreatic enzyme preparation.

6. The method of claim 1 or 2, the packaged pharmaceutical of claim 3, or the use of claim 4, wherein said agent is an inhibitor of gastric secretion.

7. The method, packaged pharmaceutical, or use of claim 6, wherein said inhibitor is an H2 histamine receptor antagonist selected from TAGAMET™ (cimetidine), ZANTAC™ (ranitidine), PEPCID™ (famotidine), or AXID™ (nizatidine).

8. The method, packaged pharmaceutical, or use of claim 6, wherein said inhibitor is an H⁺, K⁺ ATPase selected from PREVACID™ (lansoprazole), NEXIUM™ (esomeprazole magnesium), or PRILOSEC™ (omeprazole).

9. The method, packaged pharmaceutical, or use of claim 6, wherein said inhibitor is PREVACID™ (lansoprazole).

10. The method, packaged pharmaceutical, or use of claim 6, wherein said inhibitor is PROTONIX™ (pantoprazole sodium) or ACIPHEX™ (rabeprazole sodium or pariprazole).

11. The method, packaged pharmaceutical, or use of claim 6, wherein said inhibitor is a compound or a pharmaceutical composition represented by any of formulas I-XVIII or salts thereof.

12. The packaged pharmaceutical of claim 3, wherein said preparation further includes an anti-histamine.

13. The packaged pharmaceutical of claim 3, wherein said preparation further includes a decongestant.

14. The packaged pharmaceutical of claim 3, wherein said preparation further includes an anti-inflammatory agent.

15. The packaged pharmaceutical of claim 3, or the use of claim 4, for reducing the occurrence or severity of snoring.

16. The packaged pharmaceutical of claim 3, or the use of claim 4, for reducing the occurrence or severity of sleep apnea.

17. A method for conducting a medical assistance reimbursement program, comprising:
(i) providing a reimbursement program which permits, for prescription of an inhibitor of gastric secretion to reduce the symptoms or frequency of occurrence of sleep disordered breathing in a patient, at least partial reimbursement for said prescription to a healthcare provider or patient, or payment to a drug distributor for said prescription;
(ii) processing one or more claims for prescription of said inhibitor for reducing the symptoms or frequency of occurrence of sleep disordered breathing; and
(iii) reimbursing the healthcare provider or patient, or paying a drug distributor, at least a portion of the cost of said prescription.
